(19) United States
(12) Patent Application Publication
(10) Pub. No.: US 2024/0302360 A1
(43) Pub. Date: Sep. 12, 2024

(54) COMPOSITIONS AND METHODS FOR USE IN FLOW CYTOMETRY

(71) Applicant: BIOAFFINITY TECHNOLOGIES, INC., San Antonio, TX (US)

(72) Inventor: William E. Bauta, San Antonio, TX (US)

(73) Assignee: BIOAFFINITY TECHNOLOGIES, INC., San Antonio, TX (US)

(21) Appl. No.: 18/667,900

(22) Filed: May 17, 2024

Related U.S. Application Data
(63) Continuation of application No. PCT/US2022/050631, filed on Nov. 21, 2022.
(60) Provisional application No. 63/281,509, filed on Nov. 19, 2021.

(51) Int. Cl. G01N 33/533; G01N 33/547

(52) U.S. Cl. CPC G01N 33/533; G01N 33/547

(57) ABSTRACT

A compensation bead comprising an amine functionalized bead modified covalently or non-covalently with a carboxyaryl porphyrin, a method for making the amine functionalized bead with the carboxyaryl porphyrin and a method of using the amine functionalized bead with the carboxyaryl porphyrin as a marker for the carboxyaryl porphyrin labeled cells and particles.

### CROSS-REFERENCE TO RELATED APPLICATIONS

This application is continuation application of International Patent Application No. PCT/US2022/050631 filed Nov. 21, 2022, which claims priority to and benefit of the filing of U.S. Provisional Patent Application No. 63/281,509 titled “Composition and Method for Use in Flow Cytometry” filed Nov. 17, 2021, and the specification and claims thereof are incorporated herein by reference.

### STATEMENT REGARDING FEDERALLY SPONSORED RESEARCH OR DEVELOPMENT

Not Applicable.

### THE NAMES OF PARTIES TO A JOINT RESEARCH AGREEMENT

Not Applicable.

### INCORPORATION BY REFERENCE OF MATERIAL SUBMITTED ON A COMPACT DISC

Not Applicable.

### STATEMENT REGARDING PRIOR DISCLOSURES BY THE INVENTOR OR A JOINT INVENTOR

Not Applicable.

### BACKGROUND

The selective uptake and retention of hematoporhyrins in cancerous tissues has been known for many years. 1,2 Porphyrins also exhibit strong fluorescence, that is, the absorption of light of a given wavelength and subsequent emission of light at a longer wavelength, a phenomenon known as the Stokes shift. Photodynamic therapy (PDT) is a treatment approach for cancer wherein a patient is administered a porphyrin which distributes to the tumor and the tumor is subsequently irradiated with laser light of the appropriate wavelength to be absorbed by the porphyrin.³⁻⁸ In the presence of oxygen, some of the absorbed light energy is transferred from the porphyrin to oxygen molecules and results in the formation of high energy reactive oxygen species, such as singlet oxygen, which are cytotoxic to the tumor cells.^{9,10}

Combining porphyrin cancer cell selectivity and porphyrin fluorescence has produced a system and method for use of porphyrins to diagnose the presence of cancer cells in vitro and in vivo.⁹-11 The tetraaryl porphyrin TCPP is a 5,10,15,20-tetraaryl porphyrin, one of many porphyrins in this structure class. TCPP (5,10,15,20-tetrakis-(4-carboxyphenyl)-21,23H-porphine) has been known for many years in the literature and is one of several tetraaryl porphyrins that display selective uptake in cancer tissue¹².

TCPP has recently been shown to be a useful label to identify cells that are precancer or cancer cells when analyzed in a flow cytometer. Flow cytometers are instruments which analyze biological cells and particles in a thin stream of fluid intersected by an illumination source, usually a laser beam, with the resulting forward scatter (FSC) and side scatter (SSC) and fluorescent light, emitted by fluorophores on the cells or particles being analyzed with photomultiplier tubes (PMTs). Fluorescence detection channels are usually indicated by the designations F1, F2, F3, etc., depending on the number of channels in the instrument. Each fluorescence detection channel is set with barrier filters to detect a range of wavelengths characteristic of a fluorophore's fluorescence emission while filtering out all others. The channel in which a specific dye is predominantly detectable may be referred to as its primary fluorescent channel while other fluorescent channels may be designated as secondary channels.

In order to obtain accurate and reproducible results, flow cytometers must be aligned and calibrated. Several types of flow cytometry control beads are available to calibrate, standardize, and control the flow cytometer and resulting data. Flow cytometry beads are useful for 1) calibration, 2) compensation and 3) cell counting for example. The need for compensation results because the fluorophores emit light that is detected within the range of more than one detector channel. Compensation is a mathematical operation to remove this “spillover” fluorescence such that the detected fluorescence in a given channel is the result of only one dye. To accomplish this, control particles functionalized with a single dye are required. Alignment, compensation, and calibration ensure that the instrument will operate at its maximum efficiency, as well as achieving reproducibility such that data taken over time or with various instruments will be comparable.

Alignment is the process of adjusting and focusing the various optical and electrical components such that scatter and fluorescence signals are tuned to their highest intensity and tightest distribution, i.e., lowest coefficient of variation (CV) of the distribution. The components of the flow cytometer to be aligned include the laser, lenses, mirrors, barrier filters, and PMTs.

Compensation is the process of computationally removing spillover fluorescence signal from fluorescent dyes in secondary fluorescence channels due to spectral overlaps not removed by the barrier filters for the respective channels.

Calibration of a flow cytometer with proper standards ensures that the results from samples will be comparable over time and between different instruments. For the calibration of the intensity of fluorescence signals to be independent of the specific instrument and instrument settings, the excitation and emission spectra of the calibration standards and of the samples being measured must be equivalent and the measurements on each must be made under the same instrument settings.

An inherent characteristic of multicolor flow cytometry, in which multiple fluorophores are used to label (sometimes referred to as stain) cell populations, is the need to differentiate and quantify the fluorescence intensity at a given wavelength that is due to each of the fluorophores used in labeling/staining. Ideally, the fluorescence emission of each fluorophore would occur exclusively in a specific detection range of a channel and not in the detection range of other detection channels intended for other fluorophores. In practice this is usually not the case. For this reason, a methodology must be employed to remove the interfering fluorescence from other fluorophores in a detection channel. This is practically accomplished through the use of compensation standards.¹³-15

A compensation standard is typically a synthetic bead or microsphere of a size compatible with the flow cytometer. The bead is stained or otherwise functionalized with a single fluorophore type wherein the bead is covered by many individual fluorophores. The fluorescence spectrum and fluorescence intensity produced by the fluorophores on the bead is similar to the fluorescence observed when cells of interest are stained with the same fluorophore.¹⁶ Ideally the fluorophore on the bead is identical in its spectrum to the fluorophore on the cell. Many flow cytometry beads stained with common fluorophores such as rhodamine and fluorescein are commercially available. However, as of the time of filing, there is no commercial source for flow cytometry standards stained with TCPP. A TCPP conjugated bead for use in flow cytometry would be useful to perform compensation in a flow cytometer when detecting TCPP labeled cancer cells in a flow cytometer assay.

Modification of an unstained bead with a fluorophore may be achieved with non-covalent or covalent staining of the bead, which in turn is dependent on the chemical nature of the bead and the fluorophore. In the case of TCPP, each molecule has four carboxylate groups capable of covalent modification suitable for attachment to a bead, for example by formation of amide bonds between the carboxy groups and amino groups on a bead. TCPP also has an affinity for plastics such as polystyrene and will adhere to such plastics even in the absence of covalent modification. Finally, as is the case with many porphyrins, TCPP is capable of aggregation, wherein multiple TCPP molecules bind non-covalently to each other to form aggregates that can in some cases result in particulates that precipitate from solution.¹⁷ Aggregation can also affect the fluorescence characteristics of porphyrins, typically causing a decrease in such fluorescence due to fluorescence quenching, that is, the absorption of light emitted by a porphyrin molecule by a nearby porphyrin molecule.¹⁸

For successful TCPP functionalized flow cytometry compensation beads, the beads must be of a size that is compatible with the fluidics of the flow cytometer, must not settle too quickly prior to sample acquisition, and must have TCPP fluorescence (typically measured at about 650 nm) whose intensity is within the range of the detector at the voltage used in an assay, and which is comparable to the fluorescence observed in cells of interest. Now fluorescence intensity, usually described statistically by the median fluorescence intensity (MFI) of a positively stained population of beads, is a function of the inherent fluorescence of the fluorophore, the sized of the bead, and the degree of functionalization of the bead. In this context, a bead functionalized with too few TCPP molecules will have insufficient fluorescence and a bead functionalized with too many TCPP molecules could also have insufficient fluorescence due to fluorescence quenching between proximal porphyrins.

The preparation of amino-functionalized latex beads to which TCPP was covalently conjugated at its NHS ester for use in protein affinity studies was reported by Kabe et al.¹⁹-21 Kabe reported the reaction of TCPP in DMF with NHS (N-hydroxysuccinimide) to form a mono-NHS ester of one of the four carboxy groups (FIG. 1). NHS esters are known as activated esters in that the NHS group can function as a leaving group in reactions with amines, resulting in the formation of an amide bond.^{22,23} The common procedure for the formation of NHS esters is the reaction of a carboxylic acid with NHS in the presence of a peptide coupling reagent, usually one in the carbodiimide class, such as EDC (1-(3-Dimethylaminopropyl)-3-ethylcarbodiimide hydrochloride).²² Kabe reports the formation of the mono-NHS ester of TCPP (FIG. 1, 1) by reacting TCPP and one molar equivalent of NHS in DMF at room temperature for 2 h, in the absence of any peptide coupling reagent.¹⁹ Kabe went on to react the NHS ester intermediate (1) with modified latex beads containing an amino group on a linker appendage (FIG. 1, 2) to produce a covalently modified bead (FIG. 1, 3). The Kabe method was indicated to form a single amide linkage between TCPP and an amino group on the linker on the surface of the bead which would not be expected to significantly alter the absorbance or fluorescence properties of TCPP. Moreover, it would be anticipated that this type of conjugation would be chemically stable on refrigerated storage and under the experimental conditions of a flow cytometry assay. However, the reported TCPP beads of Kabe having a small diameter (2 μm) were too small to produce beads with sufficient fluorescence intensity and this limitation would make it difficult to distinguish from small particulates and debris by flow cytometry. Therefore, the Kabe method did not produce a workable solution to the problem to be solved even when larger beads were labeled with TCPP.

### BRIEF SUMMARY OF THE INVENTION

One aspect of the present invention is bead compositions modified with the porphyrin TCPP.

One aspect of the present invention is bead compositions covalently and non-covalently modified with TCPP.

Another aspect of the present invention are bead compositions modified with TCPP and having mean diameters between about 5 μm to about 20 μm, such as between about 8.0 μm and about 15 μm.

Another aspect of the invention is TCPP modified beads with utility in flow cytometry compensation.

A related aspect of the invention is TCPP modified beads for use with a TCPP lung health assay such as the CyPath® Lung assay.

A related aspect of the invention is TCPP modified beads that are stable in buffered solution for more than six months.

Another aspect of the invention are methods for the functionalization of amine-functionalized polystyrene beads with peptide coupling reagents and TCPP.

Another aspect of the invention are methods for the functionalization of amine-functionalized beads with activated esters formed in situ.

Another aspect of the invention is a method for the preparation of TCPP functionalized amine beads by non-covalent modification of amine beads.

A related aspect of the invention is a method for the modulation of the fluorescence of porphyrin functionalized beads through a filtration process.

A related aspect of the invention is a method for the reduction of TCPP-related particulates in the bead sample through a filtration process.

Another related aspect of the invention is the modulation of the fluorescence of porphyrin functionalized beads by adjusting the relative amounts of the porphyrin and the peptide coupling reagent used in the functionalization.

Another related aspect of the invention is the modulation of the fluorescence of porphyrin functionalized beads by adjusting the relative amounts of the porphyrin and the beads used in the functionalization reaction.

Another aspect of the invention is the modulation of TCPP bead fluorescence by changing the functionalization reaction time.

Another aspect of the invention are methods for the purification of beads functionalized with TCPP.

Yet another aspect of the invention are methods for the use of TCPP modified beads in multicolor flow cytometry compensation.

Yet another aspect of the invention are methods in which TCPP functionalized beads comprise a component of a method for detecting pathologies in human tissue samples.

One aspect of one embodiment of the present invention provides for an amine functionalized bead bonded with TCPP to be used as a compensation bead that can also be useful to calibrate the flow cytometer.

One embodiment of the present invention comprises a compensation bead useful as a standard for a flow cytometer. The compensation bead comprises an amine functionalized bead having a diameter of between about 3 μm to about 18 μm wherein an amine group is covalently bonded to a polymer that forms the amine functionalized bead and a 5,10,15,20-tetrakis-(4-carboxyphenyl)-21,23H-porphine (TCPP) bonded to the polymer of the amine functionalized bead in such a manner that when the amine functionalized bead bonded with TCPP is suspended in a media.

Another embodiment of the present invention comprises a compensation bead that comprises an amine functionalized bead having a diameter of between about 3 μm to about 18 μm wherein an amine group is covalently bonded to a polymer that forms the amine functionalized bead and a 5,10,15,20-tetrakis-(4-carboxyphenyl)-21,23H-porphine (TCPP) bonded to the polymer and which, in addition, contains TCPP that is non-covalently bonded to the polymer.

Another embodiment provides a method for making a compensation bead comprising the steps of reacting an amine group on a polymer that forms an amine functionalized bead with a 5,10,15,20-tetrakis-(4-carboxyphenyl)-21,23H-porphine (TCPP) having an activated ester to produce an amine functionalized bead bonded with TCPP. The amine functionalized bead bonded with TCPP is separated from the unreacted TCPP. The amine functionalized bead bonded with TCPP is collected for use as the compensation bead. This method further comprising incubating the TCPP with N-(3-dimethylaminopropyl)-N′-ethyl carbodiimide hydrochloride (EDC), N-hydroxysuccinimide (NHS), sulfo-NHS or any combination thereof in dimethylformamide (DMF) to produce the TCPP with the activated ester prior to the reacting step. In one embodiment, the TCPP with the activated ester is in a DMF solution and combined with water and filtered to produce a filtrate of the TCPP with activated ester prior to the reaction step. In another embodiment, the reacting step proceeds in a solution comprising at least one of 2-(N-morpholino)-N′-ethanesulfonic acid hemisodium salt (MES) DBPS, DMF, water, aqueous DMF or any combination thereof at a pH of between about 6 to about 7. For example, the activated ester of the TCPP is reacted with the amine group on the polymer to form an amide bond. The separating step may further comprise filtering the unreacted TCPP and the amine functionalized beads bonded with TCPP through a filter of about 5 μm porosity; and removing the amine functionalized beads bonded with TCPP from the filter.

Another embodiment provides a method for making a compensation bead comprising the steps of reacting an amine group on a polymer that forms an amine functionalized bead with a 5,10,15,20-tetrakis-(4-carboxyphenyl)-21,23H-porphine (TCPP) and EDC in a solution having one or more selected from MES buffer (pH about 6), DPBS buffer (pH about 7), DMF, and water to produce an amine functionalized bead bonded with TCPP. Further still, the EDC and the TCPP are present at a mol/mol ratio of between about 0.35 to about 25.91. The amine functionalized bead bonded with TCPP is separated from unreacted TCPP. The amine functionalized bead bonded with TCPP are collected for use as the compensation bead. The separating step may further comprise filtering the unreacted TCPP and the amine functionalized beads bonded with TCPP through a filter of about 5 μm porosity; and removing the amine functionalized beads bonded with TCPP from the filter.

According to one or more embodiments, a fluorescence spectra exhibited by the amine functionalized bead bonded with TCPP is comparable with the fluorescence spectra of TCPP dissolved in the media.

For example, the fluorescence spectra has an excitation of about 400 nm and an emission of between about 600 nm to about 800 nm. The media may be selected from Dubelco's phosphate buffered saline, (DPBS), Hank's balanced salt solution (HBBS) or a combination thereof in a pH range of between about 6.5 to about 7.5. Further still, the TCPP is bonded to the amine functionalized bead covalently, non-covalently or a combination thereof, for example, the TCPP is bonded covalently to the amine functionalized bead via an amide bond whereby the carboxy portion is present in the TCPP and the amine portion is part of the amine functionalized bead. The amine functionalized bead may be a polymer selected from polystyrene, divinylbenzene crosslinked polystyrene, latex, polyvinylamine (PVA), chitosan, poly(methyl methacrylate) (PMMA), epoxy, silica, carbon fiber, and iron oxide. The amine functionalized bead is between about 5 μm to about 20 μm, for example between about 8 μm and about 15 μm. In one embodiment, there is no SGNEGDN linker between the TCPP and the polymer that forms the amine functionalized bead. Further still, the unreacted TCPP comprise TCPP in a solution of the reacting step, TCPP derivatives formed during the reacting step or a combination thereof. In any of the embodiments, the compensation beads may have a median fluorescence intensity of between about 140 to about 1500, for example greater than about 200 or for example between about 300 to about 1300.

### DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more embodiments of the invention and are not to be construed as limiting the invention. In the drawings:

FIG. 1 illustrates the reaction sequence described by Kabe wherein TCPP reacted with NHS in DMF form an activated NHS ester (1) and subsequently reacted with latex beads bearing a terminal amino group (—NH₂) on a spacer appendage (SGNEGDN) (2) to produce TCPP functionalized beads (3).

FIG. 2 illustrates a schematic of a generalized reaction scheme between TCPP and an amine (—NH₂) functionalized polystyrene bead in the presence of a peptide coupling reagent wherein the beads are devoid of a spacer appendage such as the SGNEGDN (a divalent epoxide, ethyleneglycoldiglycidylether (EGDE), as a spacer after aminolysis of epoxy groups (SGN beads)) according to one embodiment of the present invention.

FIG. 3 illustrates a schematic of a generalized reaction scheme between an activated ester of TCPP and an amine functionalized polystyrene bead in a suitable solvent. R¹-R⁴ may represent an activated ester of a carboxylic acid or a carboxylic acid, wherein at least one R¹-R⁴ is an activated ester according to one embodiment of the present invention.

FIG. 4A-F illustrates representative dot plots and histograms derived from flow cytometry analysis of samples, wherein Spherotech 10.6 μm diameter amine beads were reacted with TCPP in the presence of 1-Ethyl-3-(3-dimethylaminopropyl)carbodiimide (EDC) and where the reagent ratio of TCPP to EDC was varied with other reaction parameters kept the same (see Table 2, 3) according to one embodiment of the present invention.

FIG. 5 illustrates the relationship between median fluorescence intensity (MFI) of TCPP functionalized amine beads and the ratio of EDC to TCPP, expressed here as a ratio of reagent volumes EDC/TCPP (v/v) according to one embodiment of the present invention. Points represent the mean of three replicate experiments and error bars represent one standard deviation from the mean.

FIG. 6 Illustrates the percentage of TCPP-related particulates, as a percentage of all events collected by flow cytometry, is shown as a function of the EDC to TCPP reagent ratio, expressed here as a ratio of reagent volumes EDC/TCPP (v/v) according to one embodiment of the present invention. Points represent the mean of three replicate experiments and error bars represent one standard deviation from the mean.

FIGS. 7A-F illustrates dot plots (FIGS. 7A-C) and histograms (FIGS. 7D-F) derived from flow cytometry analysis of samples A-D (Table 3), wherein EPRUI 10 μm diameter beads were reacted with TCPP in the presence of EDC, where the ratio of TCPP to beads was varied (Tables 3, 4) according to one embodiment of the present invention.

FIG. 8 illustrates a plot of median fluorescence intensity (MFI) of TCPP functionalized beads as a function of reagent ratio, in this case the ratio of TCPP stock solution volume to the volume of 10.6 μm diameter amine bead suspension. Points represent the mean of three replicate experiments and error bars represent one standard deviation from the mean.

FIG. 9 Illustrates the percentage of TCPP-related particulates, as a percentage of all events collected by flow cytometry, is shown as a function of the TCPP stock solution to amine bead reagent ratio, expressed here as a ratio of reagent volumes TCPP/Beads (v/v). Points represent the mean of three replicate experiments and error bars represent one standard deviation from the mean.

FIG. 10A-B Illustrates a dot plot and histogram (FIG. 10A), and a micrograph (40×) FIG. 10B corresponding to the experiment where TCPP and EDC are reacted in MES buffer in the absence of a peptide coupling reagent. Small particulates are visible in the dot plot.

FIG. 11A-B illustrates a dot plot and histogram (FIG. 11A), and a micrograph (40×) (FIG. 11B) for the reaction between TCPP and amine beads in MES buffer in the absence of a peptide coupling reagent. TCPP-related particulates are absent in the micrograph.

FIG. 12A-H illustrates Dot plot and histograms for TCPP functionalized beads from the reaction between 10 um EPRUI beads and TCPP with EDC in MES buffer under the conditions described in Table 5, conditions F, G, H, and I, respectively according to one embodiment of the present invention.

FIG. 13A-B flow cytometry results are given for TCPP-functionalized beads produced from a reaction where TCPP was first incubated with a molar excess of N-hydroxysuccinimide (NHS) and EDC in dimethylformamide (DMF) for about 5 hours, then reacted for 16 hours with 10.6 μm amine beads according to one embodiment of the present invention. FIG. 13A illustrates a Dot plot and FIG. 13B illustrates a histogram.

FIG. 14 Illustrates fluorescence spectra for: TCPP (4.94×10⁻⁶ M) in HBSS (solid line), TCPP-functionalized beads in HBSS (hatched line), and A549 lung cancer cells stained with TCPP in HBSS (dotted line) illustrate overlapping/comparable fluorescent spectra according to one embodiment of the present invention. Spectra of beads and cells were normalized relative to fluorescence maximum on TCPP solution spectrum by dividing fluorescence values factors of 0.95 and 2.27, respectively. Spectra collected on a Tecan Spark plate reader using 100 μL per well, with an excitation wavelength of 410 nm and fluorescence detection 600-800 nm.

FIG. 15 illustrates a schematic representation of the bidirectional filtration flowchart process according to one embodiment of the present invention. Step A, a plunger is removed from a disposable plastic syringe. Step B, a filter is attached to the syringe. Step C, the syringe is charged with HBSS (4 mL). Step D, diluted bead suspension is added to the syringe and resuspended. Step E, a plunger is replaced and the bead suspension slowly filtered. Filtrate is discarded. Step F, fresh HBSS is slowly sucked back through the filter into the syringe. Step G. bead suspension is slowly filtered (as before) and filtrate is discarded. Step H, beads are removed from the filter. Fresh HBSS is slowly sucked into the syringe through the filter to the 5 mL mark. The filter is removed and the bead suspension in the syringe is ejected into a collection vessel. The filter is replaced onto the syringe and the process repeated a total of five times. In one embodiment the beads are labeled with TCPP and the filtration process separates any TCPP particulates from the beads that are retained on the filter as the TCPP particulates pass through the filter and remain in the filtrate.

FIG. 16A-B illustrates micrographs of FIG. 16A TCPP-functionalized 10.6 um beads produced by the reaction of unstained amine beads with TCPP and EDC in MES at room temperature and FIG. 16.B illustrating the same beads after undergoing the bidirectional filtration process according to one embodiment of the present invention.

FIG. 17A-D illustrates Dot plots and histograms for TCPP-functionalized 10.6 μμm beads produced by the reaction of unstained amine beads with TCPP and EDC in MES at room temperature according to one embodiment of the present invention. FIG. 17A illustrates A Dot plot of 10.6 μm TCPP-functionalized beads produced by the reaction of unstained amine beads with TCPP and EDC in MES at room temperature prior to filtration. FIG. 17B illustrates A Dot plot of the same beads after bidirectional filtration. FIG. 17C illustrates A histogram of the beads described in FIG. 17A. FIG. 17D illustrates a histogram of the beads described in FIG. 17B.

FIG. 18A-B illustrates Plots of median fluorescence intensity (MFI) for TCPP-functionalized beads stored at 0-8° C. in an amber vial over a period of 300 days. Dots show relative MFI for each time point calculated as: [MFI(t)-MFI(t0)]/MFI(t0), expressed as a percentage, where MFI(t) is the MFI at time t and MFI(t0) is the initial MFI of the sample at time 0. The solid horizontal bars are at 90% and 110% relative to the time zero MFI. FIG. 18B illustrates particulate change percentage as compared to initial levels at time zero.

FIG. 19 illustrates an NHS-ester structure of TCPP wherein each R is selected from: H, SO₃H, and SO₃⁻M⁺ wherein the structure forms from TCPP in the presence of molar excess of NHS or Sulfo-NHS and EDC according to one embodiment of the present invention.

FIG. 20 illustrates a reaction of TCPP (1) with a molar excess of NHS (or Sulfo-NHS) and EDC to produce the tetra NHS ester (2) and EDC-related soluble byproduct (3) according to one embodiment of the present invention.

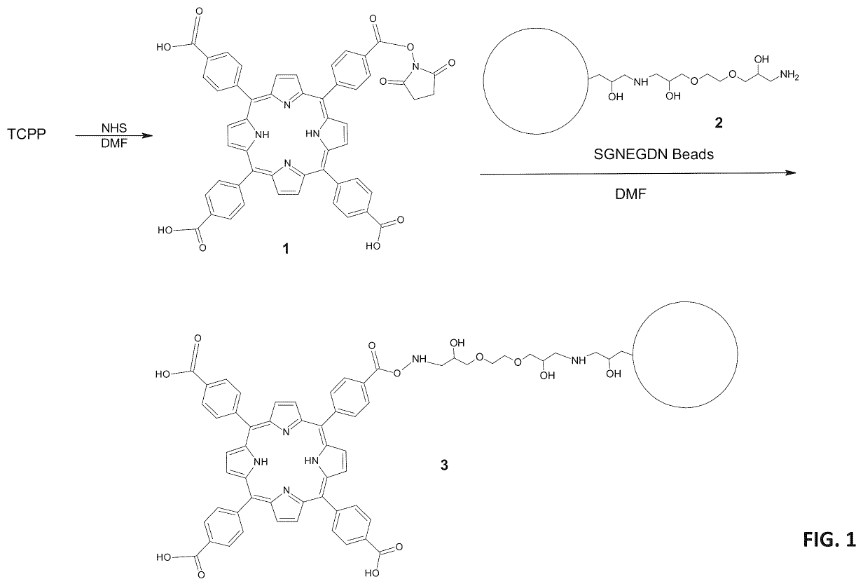

### DETAILED DESCRIPTION OF THE INVENTION

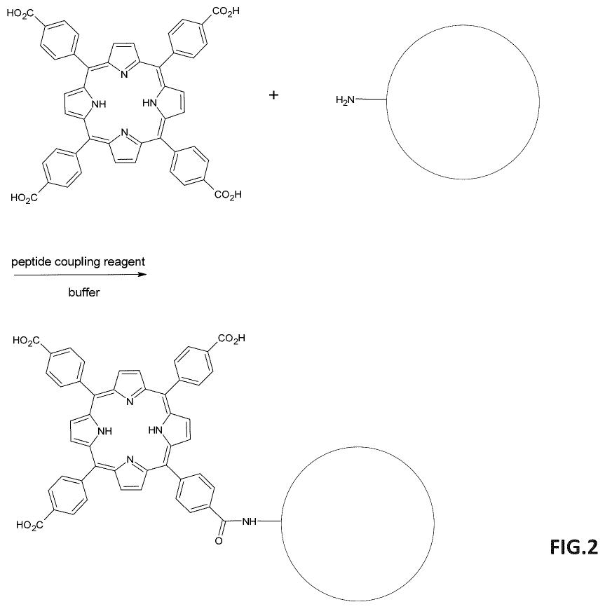

One or more embodiments of the present invention describe compositions comprised of synthetic beads modified covalently or non-covalently with TCPP and methods for producing the same. One preparation of these beads is illustrated in FIG. 2. Commercial amine functionalized polystyrene (divinylbenzene crosslinked) beads of ˜10 um diameter were suspended in pH 4-6 aqueous buffer in a plastic tube with a cap and treated with a solution of TCPP, prepared either in water containing sodium bicarbonate or in a mixture of water and isopropanol with sodium bicarbonate. To this mixture was added a peptide coupling reagent. The tube is capped and the mixture is agitated/mixed in an inverting agitator overnight (12-20 h) with the exclusion of light. The bead suspension was then centrifuged, and the supernatant solution aspirated. Beads were then resuspended in phosphate buffer (DPBS) or Hank's balanced salt solution (HBSS). Thereafter, the beads are stored under refrigeration (2-10° C.) in a plastic tube with the exclusion of light. (See EXAMPLE 4, 5, 6,16).

Initial results demonstrated that a 10 μm diameter bead stained with TCPP might have the right combination of fluorescence and hydrodynamic properties for flow cytometry. Thus, amine functionalized polystyrene beads with an about 10.6 μm diameter fluorophore labeled/stained in aqueous media with a solution of TCPP at room temperature, in the absence of a peptide coupling reagent, produced fluorescent beads with a median fluorescence intensity (MFI) of 140. A MFI of 140 was too low for the TCPP labeled beads to serve as a compensation standard for TCPP labeled sputum cells for use in a flow cytometric assay but may be useful as a compensation standard in other non-flow cytometric assay applications. The Kabe procedure was varied wherein TCPP was treated with NHS in DMF, followed by reaction with the 10.6 μm beads but there was no significant improvement in bead MFI compared to TCPP alone. The Kabe procedure was further modified wherein TCPP was incubated in DMF with one molar equivalent each of NHS and EDC, in an attempt to generate a mono-NHS ester intermediate, followed by treatment with 10.6 μm diameter amine beads. The resulting beads also had a disappointing MFI (154). The reaction with TCPP and EDC (1 molar equiv. each in DMF) in the presence of the beads without pre-incubation also resulted in poor staining (MFI 96.2).

The direct reaction between amine functionalized polystyrene beads and TCPP using the peptide coupling reagent EDC in pH 6 aqueous buffer is described herein according to one embodiment of the present invention. The reaction produced beads with acceptable fluorescence, hydrodynamic properties, and excellent stability on storage. The reaction also produced a side reaction between TCPP and EDC that results in a TCPP-related particulate that is unrelated to the beads. The amount of this particulate that forms is a function of the reagent ratios (TCPP, EDC, beads) and the reaction conditions. To address the particulate produced in the reaction, a filtration procedure capable of reducing the amount of TCPP-related particulate that is produced in addition to the TCPP labeled beads was developed. Further, TCPP labeled beads with sufficient fluorescence for the utility of a cytometric assay were developed by reacting TCPP with EDC and NHS, both in large molar excess in DMF, and by adding 10.6 μm diameter amine beads.

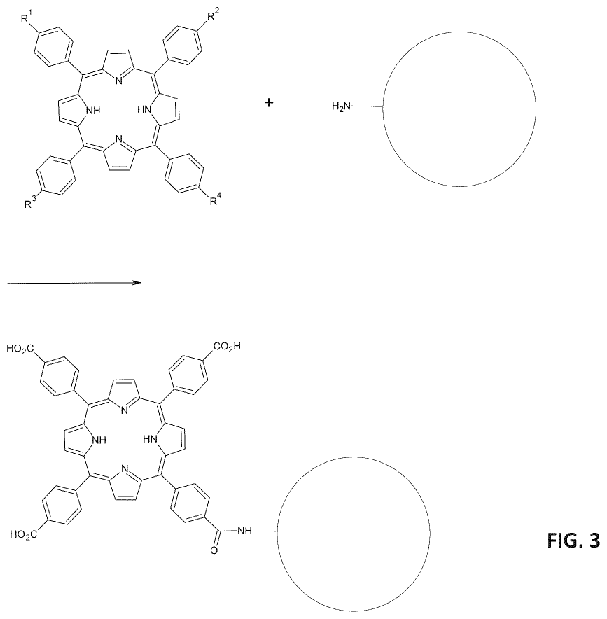

In another embodiment, preparation of TCPP functionalized beads is described. To a solution of TCPP in an organic solvent, such as DMF, is added a reagent suitable for the preparation of an activated carboxylate ester, such as N-hydroxysuccinimide, and a peptide coupling reagent and mixed for a time, for example for over 4 hours to form an activated ester in situ (FIG. 3). This in situ activated ester or esters of TCPP is then reacted with amine functionalized polystyrene beads (for example a bead of about 10.6 μm) in a rotating agitator overnight. The bead suspension was then centrifuged, and the supernatant solution aspirated. Beads were then resuspended in phosphate buffer (DPBS) or Hank's balanced salt solution (HBSS). Such beads are then stored under refrigeration (2-10° C.) in a plastic tube with the exclusion of light. (See EXAMPLE 9 for a discussion on a TCPP NHS ester bead staining procedure)

In another embodiment of the present invention, a method is described in which the beads used to react with TCPP in the presence of a peptide coupling reagent in a suitable buffer have a mean diameter of between about 8 to about 13 μm, and are functionalized with amino groups. (See EXAMPLE 4, 5, 6,16)

Another embodiment of the present invention provides for TCPP functionalized beads with a diameter of between 8 μm and 15 μm. (See EXAMPLE 4, 5, 6,16)

In another more specific embodiment of the invention, we describe the beads used to react with TCPP as spherical amine functionalized polystyrene beads with a mean diameter of 10-11 μm and bearing 7×10¹¹ amine groups per bead. For example, Spherotech beads catalog APX-100-10. (See EXAMPLE 4, 5, 6)

In another embodiment of the present invention, a method to produce functionalized TCPP beads is described wherein beads used to react with TCPP are spherical amine functionalized polystyrene beads with a mean diameter of between about 10 μm to about 11 μm and bearing approximately 2×10⁻⁶ moles of amine groups per mg of beads. (See EXAMPLE 4 for a general TCPP/EDC bead staining procedure)

In another more specific embodiment of the invention, we describe the beads used to react with TCPP as spherical amine functionalized polystyrene beads with a mean diameter of between about 10 μm to about 11 μm and bearing approximately 1×10⁻⁷ moles of amine groups per mg of beads is provided. For example, EPRUI beads are the beads used. (See EXAMPLE 4 a general TCPP/EDC bead staining procedure)

In yet another embodiment of the present invention, a method for preparation of TCPP functionalized beads entails a reaction between TCPP and amine functionalized beads carried out with a peptide coupling reagent under buffered conditions of PH ˜6 using ˜0.1 N aqueous MES ((2-(N-morpholino)-N′-ethanesulfonic acid hemisodium salt) buffer is provided. (See EXAMPLE 4 a general TCPP/EDC bead staining procedure)

In yet another embodiment of the present invention, a method for preparation of TCPP functionalized beads entails a reaction between TCPP and amine functionalized beads using a carbodiimide peptide coupling reagent in a pH 4-6 buffer is provided. (See EXAMPLE 4)

In a further embodiment of the present invention, a method for preparation of TCPP functionalized beads entails reaction between TCPP and amine functionalized beads using the peptide coupling reagent EDC (N-(3-dimethylaminopropyl)-N′-ethyl carbodiimide hydrochloride) in a pH 4-6 buffer is provided. (See EXAMPLE 4 a general TCPP/EDC bead staining procedure)

In another embodiment of the present invention, a method for preparation of TCPP functionalized beads entails reaction of TCPP, peptide coupling reagent EDC (N-(3-dimethylaminopropyl)-N′-ethyl carbodiimide hydrochloride), and N-hydroxysuccinimide in DMF for 2-6 h, followed by addition of amine functionalized beads as an aqueous suspension (FIG. 3) is provided. (See EXAMPLE 9 for a TCPP NHS ester bead staining procedure)

In another embodiment of the present invention, a method for preparation of TCPP functionalized beads entails reaction between TCPP and the amine functionalized beads using a peptide coupling reagent for example EDC (N-(3-dimethylaminopropyl)-N′-ethyl carbodiimide hydrochloride) in 0.1 N aqueous MES ((2-(N-morpholino)-N′-ethanesulfonic acid hemisodium salt) buffer at pH˜6 is provided. (See EXAMPLE 4, 5, 6,16,8,7)

In another embodiment of the present invention, a method for the preparation of TCPP functionalized beads entails reaction between an in situ prepared solution of an N-hydroxysuccinimide ester (or esters) of TCPP and the amine functionalized beads is provided. (See EXAMPLE 9 for a TCPP NHS ester bead staining procedure)

Yet another embodiment of the invention provides for a method for the preparation of a TCPP NHS (N-hydroxysuccinimide) ester or esters of TCPP with a molar excess of N-hydroxysuccinimide (80 eq) and EDC (23 mol eq) in DMF, and subsequent reaction of this activated ester with a commercial solution of Spherotech amine polystyrene beads at room temperature (FIG. 3). (See EXAMPLE 9 for a TCPP NHS ester bead staining procedure)

Without being overly constrained by theory, it is understood that TCPP contains four carboxylate groups and that each of these groups is capable of forming an activated ester, for example, through reaction with NHS in the presence of a peptide coupling reagent such as EDC. It is further proposed that under conditions where a large molar excess of both NHS and EDC are present, it is possible to form multiple NHS esters of TCPP, including the tetra-NHS ester of TCPP.

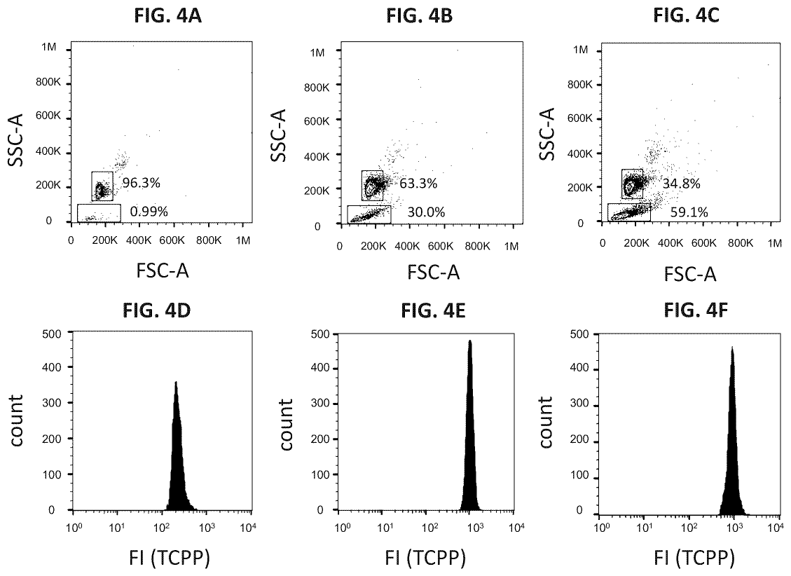

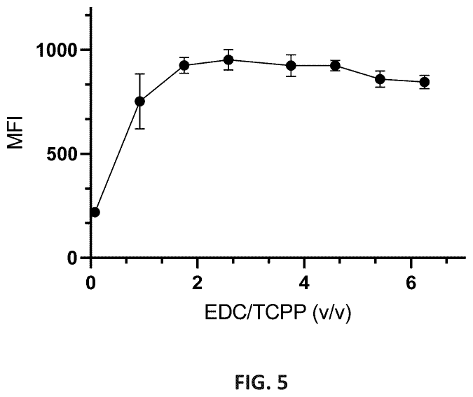

In a yet another embodiment of the present invention, a method is disclosed for modulation of TCPP-amine bead fluorescence in which an aqueous suspension of between about 10 to about 11 μm diameter spherical amine functionalized beads is reacted in an inverting agitator overnight with TCPP and EDC in variable reagent proportions as described in Table 1 (Samples A-H) and the bead suspensions subsequently centrifuged, the supernatant aspirated and bead pellets resuspended in HBSS (0.800 mL each) and stored at 4° C. in the dark. The bead staining experiment was run three times. Median fluorescence intensity (MFI) values for each experiment (MFI1, MFI 2, MFI 3) and mean MFI values measured in the FL6 channel are shown in TABLE 2 and plotted FIG. 5. Representative dot plots and histograms are given in FIG. 4. (See EXAMPLE 5 for discussion of a procedure for variable EDC-TCPP ratio experiments). Referring now to FIG. 4, FIG. 4A Dot plot corresponds to Table 1 Sample A. FIG. 4B Dot plot corresponds to Table 1 Sample E. FIG. 4C Dot plot corresponds to Table 1 Sample H. FIG. 4D Histogram corresponds to Table 1 Sample A. FIG. 4E. Histogram corresponds to Table 1 Sample E. FIG. 4F Histogram corresponds to Table 1 Sample H. FIG. 4A-C illustrate Dot plots which show all the events collected from the samples, with the positive population (TCPP functionalized beads) shown in a box that represents gating, or mathematical segregation, of that population. The number next to the gated population represents the percentage of all events that the population in each box constitutes relative to the entire sample. Dot plot axes show side scattering (y axis) versus forward scattering (x-axis). The corresponding histograms FIG. 4D-F illustrate fluorescence intensity results for events in this positive population in the FL6 channel, which measures fluorescence emission from TCPP, on the horizontal axis versus the number (count) of events on the vertical axis. All data collected on Navios EX flow cytometer.

TABLE 1

| Sample | A | B | C | D | E | F | G | H |
|---|---|---|---|---|---|---|---|---|
| EDC (mL) | 0.10 | 1.10 | 2.10 | 3.10 | 4.50 | 5.50 | 6.50 | 7.50 |
| EDC (mmol) | 5.22 × 10⁻⁴ | 5.75 × 10⁻³ | 1.10 × 10⁻² | 1.62 × 10⁻² | 2.35 × 10⁻² | 2.87 × 10⁻² | 3.40 × 10⁻² | 3.92 × 10⁻² |
| Spherotech | 0.288 | 0.288 | 0.288 | 0.288 | 0.288 | 0.288 | 0.288 | 0.288 |
| beads (mL) | | | | | | | | |
| TCPP stock | 1.200 | 1.200 | 1.200 | 1.200 | 1.200 | 1.200 | 1.200 | 1.200 |
| (mL) | | | | | | | | |
| TCPP (mmol) | 1.51 × 10⁻³ | 1.51 × 10⁻³ | 1.51 × 10⁻³ | 1.51 × 10⁻³ | 1.51 × 10⁻³ | 1.51 × 10⁻³ | 1.51 × 10⁻³ | 1.51 × 10⁻³ |
| MES (mL) | 8.712 | 7.712 | 6.712 | 5.712 | 4.312 | 3.312 | 2.312 | 1.312 |
| Total Volume | 10.3 | 10.3 | 10.3 | 10.3 | 10.3 | 10.3 | 10.3 | 10.3 |
| EDC/TCPP | 0.083 | 0.917 | 1.750 | 2.583 | 3.750 | 4.583 | 5.417 | 6.250 |
| (v/v) | | | | | | | | |
| EDC/TCPP | 0.35 | 3.80 | 7.26 | 10.71 | 15.55 | 19.00 | 22.46 | 25.91 |
| mol/mol | | | | | | | | |

EDC sol.: 5.22 × 10⁻³ M in 0.10N MES buffer.
TCPP stock sol: 1.26 × 10⁻³ M in isopropanol water (1:1)
Bead susp.: spherical amine functionalized polystyrene (divinylbenzene crosslinked), 10.6 μm mean diameter, 2.5 w/v suspension.
Spherotech, Inc. (Catalog APX-100-10, Lot AM01).

TABLE 2

| EDC/TCPP | | | | |
|---|---|---|---|---|
| (v/v) | MFI 1 | MFI 2 | MFI 3 | Mean |
| 0.08 | 227 | 212 | 218 | 219 |
| 0.92 | 882 | 617 | 757 | 752 |
| 1.75 | 956 | 937 | 882 | 925 |
| 2.58 | 984 | 978 | 896 | 953 |
| 3.75 | 902 | 984 | 888 | 925 |
| 4.58 | 928 | 947 | 898 | 924 |
| 5.42 | 904 | 837 | 837 | 859 |
| 6.25 | 866 | 862 | 808 | 845 |

ND = not determined
MFI measured in the FL6 channel of a Navios flow cytometer.

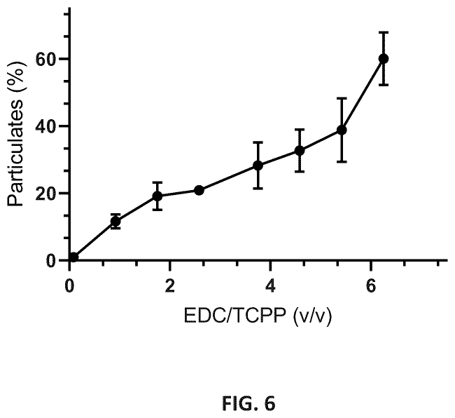

In another embodiment, a method for the modulation of the amount of TCPP-related particulates in a TCPP-functionalized bead sample is disclosed by selecting the EDC to TCPP reagent ratio (FIG. 6), using the reagent ratios described in Table 1. (See EXAMPLE 5 for discussion of a procedure for variable EDC-TCPP ratio experiments).

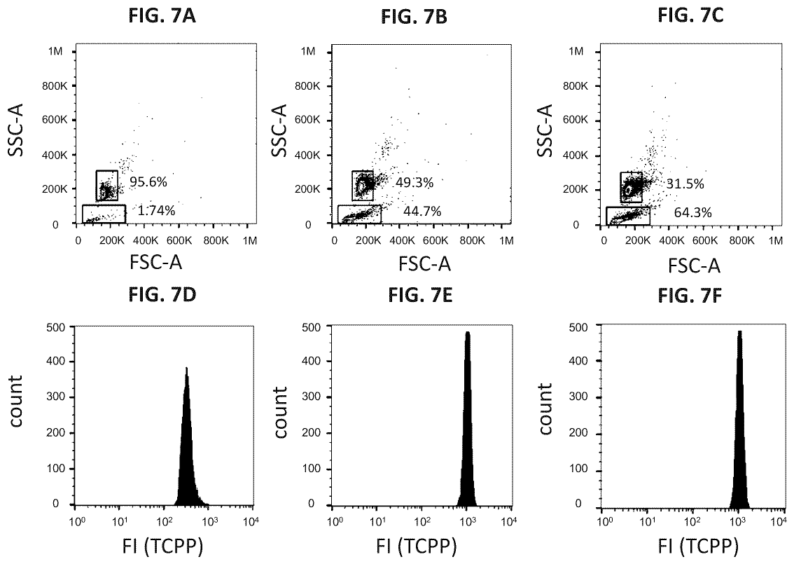

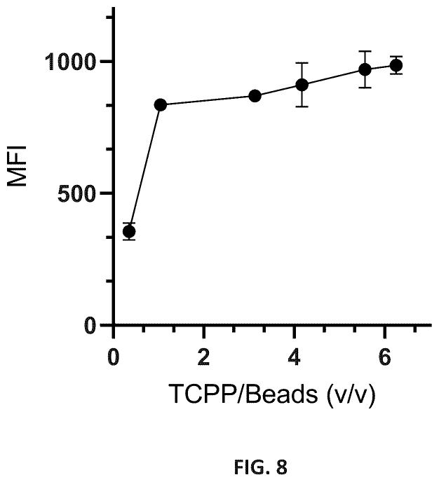

In a separate embodiment, we disclose a method for the modulation of the fluorescence of TCPP-amine beads in which an aqueous suspension of between about 10 to about 11 μm diameter spherical amine functionalized beads is reacted in an inverting agitator overnight with TCPP and beads in the volumetric reagent proportions described in Table 3 (Samples A-F) and the bead suspensions subsequently centrifuged, the supernatant aspirated and bead pellets resuspended in HBSS (800 mL each) and stored at 4° C. in the dark. MFI values for these samples are given in Table 4 and plotted in FIG. 8. Representative dot plots and histograms are given in FIG. 7 wherein EPRUI 10 μm diameter beads were reacted with TCPP in the presence of EDC, where the ratio of TCPP to beads was varied (Tables 3, 4). (See EXAMPLE 6 for discussion on a procedure for variable TCPP-bead ratio). Referring now to FIG. 7, FIG. 7A Dot plot corresponds to Table 3 Sample A. FIG. 7B Dot plot corresponds to Table 3 Sample D. FIG. 7C Dot plot corresponds to Table 3 Sample H. FIG. 7D Histogram corresponds to Table 3 Sample A. FIG. 7E Histogram corresponds to Table 3 Sample D. FIG. 7F Histogram corresponds to Table 3 Sample F. The ratio of EDC to TCPP used in each run was held constant. FIG. 7A-C Dot plots show all the events collected from the samples, with the positive population (TCPP functionalized beads) shown in a box that represents gating, or mathematical segregation, of that population. The number next to the gated population represents the percentage of all events that this population constitutes relative to the entire sample. Dot plot axes show side scattering (y axis) versus forward scattering (x-axis). The corresponding histograms FIG. 7D-F show fluorescence intensity results for events in this positive population in the FL6 channel, which measures fluorescence emission from TCPP, on the horizontal axis versus the number (count) of events on the vertical axis. All data collected on Navios EX flow cytometer.

TABLE 3

| Sample | A | B | C | D | E | F |
|---|---|---|---|---|---|---|
| EDC (mL) | 0.195 | 0.585 | 1.755 | 2.341 | 3.121 | 3.511 |
| EDC mmol | 1.95 × 10⁻³ | 5.85 × 10⁻³ | 1.76 × 10⁻² | 2.34 × 10⁻² | 3.12 × 10⁻² | 3.51 × 10⁻² |
| Sphero beads | 0.288 | 0.288 | 0.288 | 0.288 | 0.288 | 0.288 |
| (mL) | | | | | | |
| TCPP stock (mL) | 0.100 | 0.300 | 0.900 | 1.200 | 1.600 | 1.800 |
| TCPP mmol | 1.26 × 10⁻⁴ | 3.78 × 10⁻⁴ | 1.13 × 10⁻³ | 1.51 × 10⁻³ | 2.02 × 10⁻³ | 2.27 × 10⁻³ |
| MES (mL) | 9.817 | 9.427 | 8.257 | 7.671 | 6.891 | 6.501 |
| Total Volume | 10.300 | 10.300 | 10.300 | 10.300 | 10.300 | 10.300 |
| EDC/TCPP | 15.5 | 15.5 | 15.5 | 15.5 | 15.5 | 15.5 |
| (mol/mol) | | | | | | |
| TCPP/amine (v/v) | 0.35 | 1.04 | 3.13 | 4.17 | 5.56 | 6.25 |

TCPP stock solution: 1.26 × 10⁻³ M
EDC solution: 1.00 × 10⁻² M

TABLE 4

| TCPP-Beads | | | | |
|---|---|---|---|---|
| (v/v) | MFI 1 | MFI 2 | MFI 3 | Mean |
| 0.35 | 319 | 368 | 379 | 355 |
| 1.04 | 833 | 849 | 824 | 835 |
| 3.13 | 856 | 870 | 882 | 869 |
| 4.17 | 1007 | 856 | 872 | 912 |
| 5.56 | 998 | 1020 | 890 | 969 |
| 6.25 | 1016 | 989 | 950 | 985 |

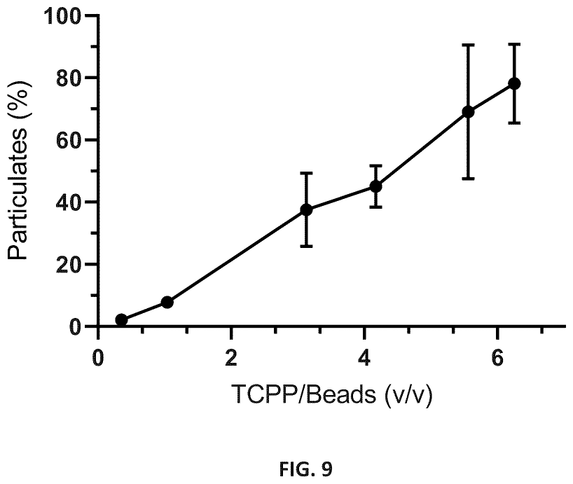

In a closely related embodiment, we disclose a method for the modulation of the amount of TCPP-related particulates in a TCPP-functionalized bead sample by selecting the TCPP to beads reagent ratio (FIG. 9), using the reagent ratios described in Table 3. (See EXAMPLE 6 for discussion of a procedure for variable TCPP-bead ratio).

In a separate specific embodiment, a method is described where amine polystyrene beads from a different source (EPRUI, 10 μm diameter) were functionalized with TCPP and EDC in 0.10 N MES buffer over a range of reagent stoichiometries as shown in Table 5 (Samples F-I). The median fluorescence intensity of these samples in the FL6 channel of the flow cytometer is give in the last row of Table 5. Reagents were combined in a plastic tube with cap and allowed to react in an inverting agitator overnight, followed by aspiration of the supernatant and resuspension of the beads in HBSS, followed by storage in in plastic vials at 4° C. in the dark. Samples were tested on a Navios flow cytometer and gave the MFI values indicated in the final row of TABLE 5. Dot plots and histograms for these samples are shown in FIG. 3A-D wherein FIG. 3A corresponds to sample F, FIG. 3B corresponds to sample G, FIG. 3C corresponds to sample H, and FIG. 3D corresponds to sample I. (See EXAMPLE 7 for a discussion of a general TCPP/EDC bead staining procedure)

TABLE 5

| Sample | F | G | H | I |
|---|---|---|---|---|
| EDC sol. (mL) | 4.519 | 3.615 | 2.711 | 1.807 |
| Bead (mg) | 10.0 | 10.0 | 10.0 | 10.0 |
| TCPP sol. (mL) | 1.20 | 0.96 | 0.72 | 0.48 |
| MES 0.10N | 5.00 | 6.15 | 7.29 | 8.43 |
| (mL) | | | | |
| Total Volume | 10.7 | 10.7 | 10.7 | 10.7 |
| MFI (FL6) | 1160 | 1200 | 1252 | 1328 |

EDC sol.: 5.20 × 10⁻³M in 0.10N MES buffer.
Beads.: spherical amine functionalized polystyrene (divinylbenzene crosslinked), 10.0 μm mean diameter, 1.0 × 10⁻⁷ mol amine/mg beads. EPRUI Biotech, Inc. (Catalog 1-003-10). TCPP (1.26 × 10⁻³M aq). MFI measured in the FL6 channel of a Navios flow cytometer.

In another specific embodiment we describe methods for the modulation of the fluorescence of TCPP functionalized beads. Reaction of amine functionalized polystyrene beads with TCPP in the absence of a peptide coupling reagent resulted in fluorescence that was approximately on tenth of that observed using a peptide coupling reagent, based on three replicate experiments (Table 6). (See EXAMPLE 15 for a reaction between TCPP and beads without EDC)

TABLE 6

| Sample | J | K | L |
|---|---|---|---|
| EDC sol. (mL) | 0.000 | 0.000 | 0.000 |
| Bead susp. (mL) | 0.288 | 0.288 | 0.288 |
| TCPP sol. (mL) | 1.200 | 1.200 | 1.200 |
| MES 0.10N | 8.800 | 8.800 | 8.800 |
| (mL) | | | |
| Total Volume | 10.288 | 10.288 | 10.288 |
| MFI (FL6) | 138 | 140 | 142 |

Bead susp.: spherical amine functionalized polystyrene (divinylbenzene crosslinked), 10.6 μm mean diameter, 1.80 × 10⁻⁶ mol amine/mg beads, 2.5 w/v suspension. Spherotech, Inc. (Catalog APX-100-10). TCPP (1.26 × 10⁻³M aq). MFI measured in the FL6 channel of a Navios EX flow cytometer.

Disclosed herein according to one embodiment of the present invention is a method by which a sequential filtration procedure can be performed on TCPP functionalized beads that systematically reduces the fluorescence of those beads. Thus, TCPP functionalized 10 μm diameter beads were resuspended in HBSS and passed through a 5 μm porosity glass syringe filter (Finneran, 5 μm, 13 mm diameter). The filtration was performed “bidirectionally,” that is, after the initial filtration of the bead suspension, fresh HBSS was sucked through the glass filter and pushed through the filter again. This process is illustrated in FIG. 5. The beads were then removed from the filter by sucking HBSS into the syringe through the filter, removal of the filter from the syringe, and dispensing the bead suspension into a plastic tube.

Without being overly constrained by theory, we surmise that the beads are functionalized both covalently and non-covalently by TCPP, that is, some of the TCPP is bound to the bead through formation a covalent, presumably an amide bond, between a carboxylate on TCPP and an amino group on the bead. In addition, there are TCPP molecules bound to the bead non-covalently, perhaps through hydrophobic or pi-stacking interactions between the aromatic portion of TCPP and the aromatic polystyrene polymer of the bead. Moreover, the possibility of porphyrin aggregation on the bead cannot be discounted. We speculate that the loss of MFI upon filtration is related to the loss of non-covalently bound TCPP from the beads.

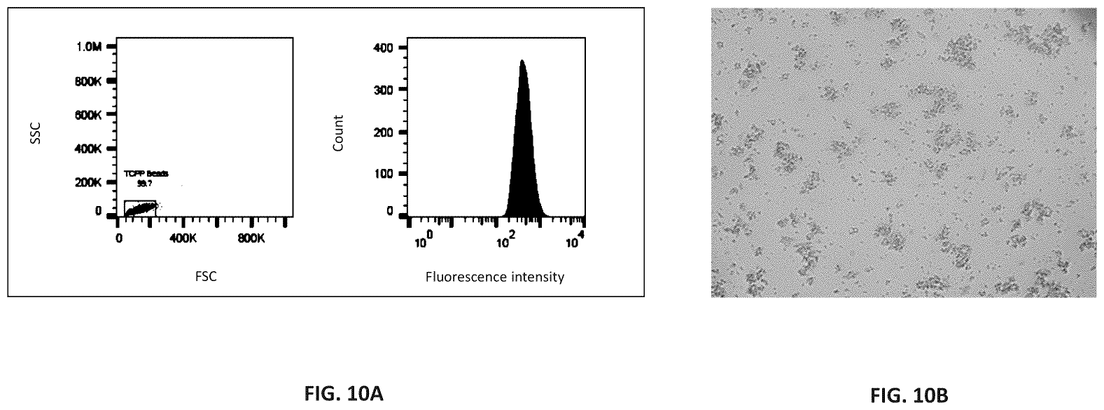

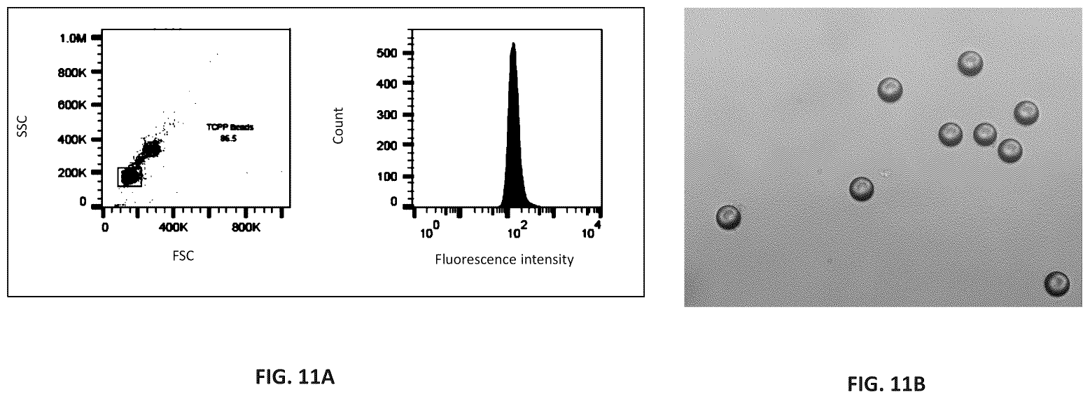

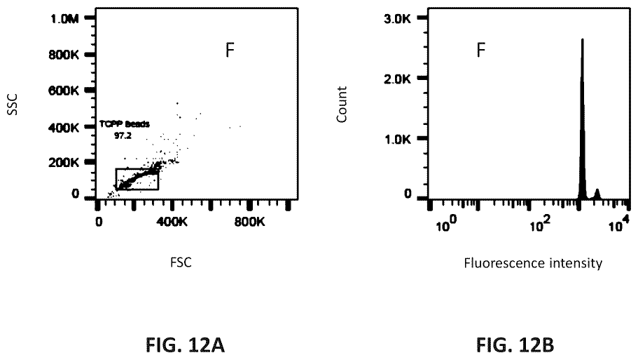

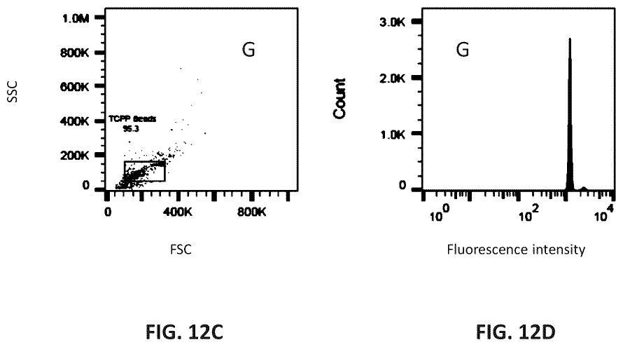

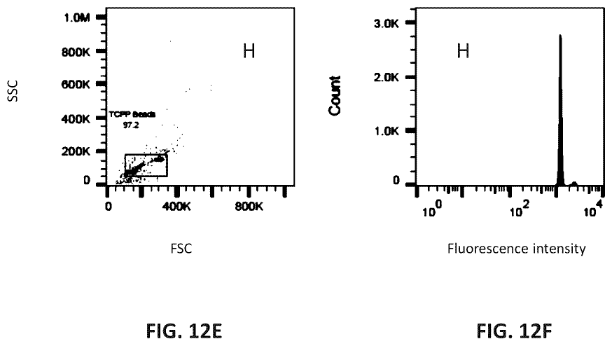

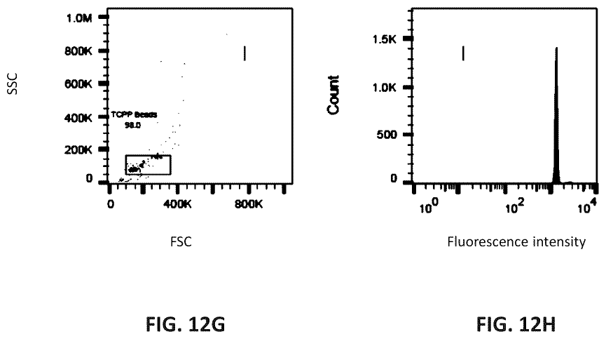

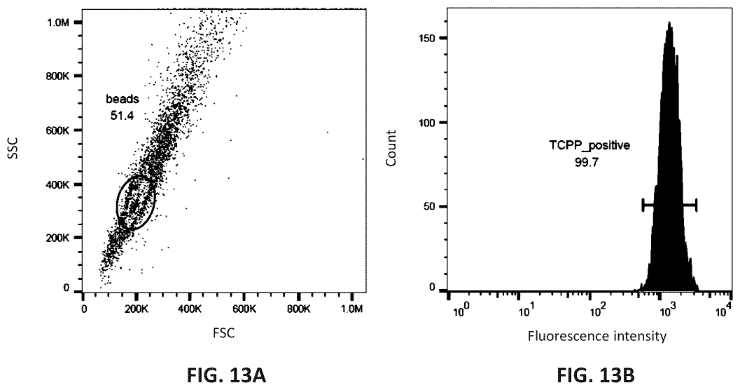

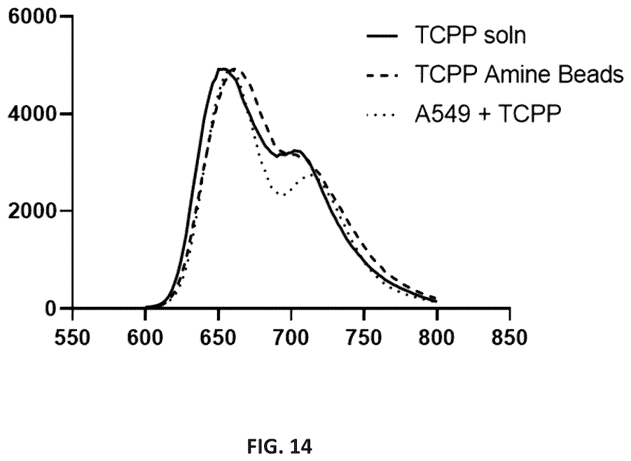

In another embodiment, a method for the purification of TCPP functionalized beads is disclosed such that non-bead fluorescent particulates are removed through a filtration process. In the presence of a peptide coupling reagent EDC, TCPP forms small particulates whose chemical structure is not understood. Without being constrained by theory, it is surmised that these particulates consist of aggregates of a TCPP derivative generated by reaction of TCPP with EDC in the absence of an amine coupling partner. Thus, TCPP-related particulates are generated when the bead functionalization conditions are performed in the absence of beads (Table 6). The particulates formed are observable both by flow cytometry (FIGS. 10A-B) and by microscopy (FIG. 10B) and are small, generally less than 5 μm in diameter, and irregular in shape. Similar small particulates are observable when the amine beads are functionalized by TCPP in the presence of EDC and may be considered a side reaction that accompanies bead functionalization. A quantitation of the amount of small particulates relative to the TCPP functionalized bead population may be derived by gating the TCPP functionalized bead population, that is, the fluorescent positive population, and determining the percentage of total events that are comprised by this positive population.

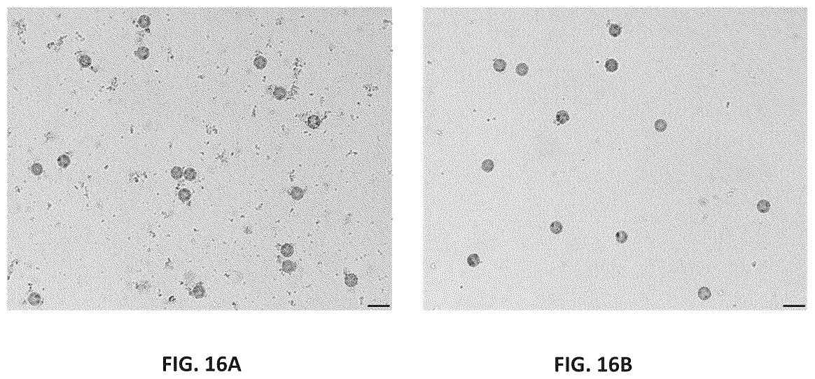

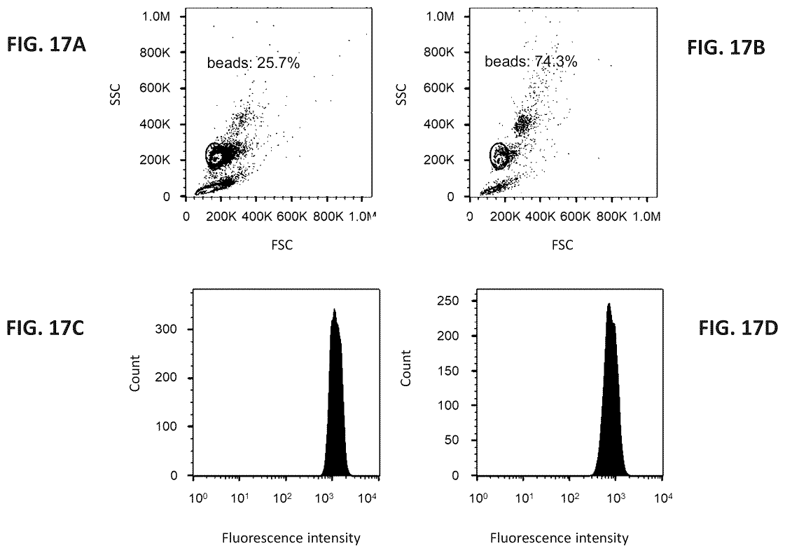

Further still, we disclose according to another embodiment of the present invention, a method for the purification of TCPP functionalized beads such that the amount of TCPP-related non-bead particulates is decreased. The bidirectional filtration process can remove small TCPP-related particulates. The marked increase in the percentage of positive events (pos. % of total) for the filtered samples is consistent with the removal of small TCPP-related particulates by filtration. This is also evident by microscopy, where small particulates are diminished in a filtered sample relative to the parent unfiltered sample (FIG. 16A-B). (See EXAMPLE 10 for a filtration procedure)

In another embodiment, a method for preparation of TCPP functionalized beads entails reaction between TCPP and the amine functionalized beads carried out using an activated ester peptide coupling reagent.

In yet another embodiment, a method for preparation of TCPP functionalized beads entails reaction between TCPP and amine functionalized beads carried out using an acyl azide peptide coupling reagent.

In another embodiment, the method for preparation of TCPP functionalized beads entails reaction between TCPP and the amine functionalized beads carried out using a phosphonium salt peptide coupling reagent.

In another embodiment, a method for preparation of TCPP functionalized beads entails reaction between TCPP and the amine functionalized beads carried out using an organophosphorus peptide coupling reagent.

In another embodiment, a method for preparation of TCPP functionalized beads entails reaction between TCPP and the amine functionalized beads carried out using a triazine peptide coupling reagent.

Another embodiment describes TCPP functionalized beads suitable for flow cytometry experiments that utilize TCPP as a cell staining reagent wherein the TCPP labeled cells has an MFI of between about 200-20000 and the TCPP functionalized beads used for flow cytometry compensation have an MFI that overlaps with the MFI of the TCPP labeled cells.

In another embodiment methods that use TCPP functionalized beads in flow cytometry experiments intended to diagnose lung cancer is provided.

In another embodiment methods that use TCPP functionalized beads in flow cytometry experiments intended to ascertain lung health is provided.

In another embodiment methods that use TCPP functionalized beads in flow cytometry experiments that analyze an ex-vivo human tissue sample is provided.

In another embodiment methods that use TCPP functionalized beads in flow cytometry experiments that analyze an ex-vivo human tissue sample consisting of sputum is provided.

In another embodiment methods that use TCPP functionalized beads in flow cytometry experiments that analyze an ex-vivo human tissue sample consisting of a cervical swab is provided.

In another embodiment methods that use TCPP functionalized beads in flow cytometry experiments that analyze an ex-vivo human tissue sample consisting of urine is provided.

In another embodiment methods that use TCPP functionalized beads in flow cytometry experiments that analyze an ex-vivo human tissue sample consisting of blood is provided.

In another embodiment methods that use TCPP functionalized beads in flow cytometry experiments that analyze an ex-vivo human tissue sample consisting of an oral or nasal swab is provided.

In another embodiment methods that use TCPP functionalized beads in flow cytometry experiments that analyze an ex-vivo human tissue sample consisting of feces is provided.

According to one aspect of the present invention, a method wherein synthetic beads containing amino groups are modified with TCPP is provided.

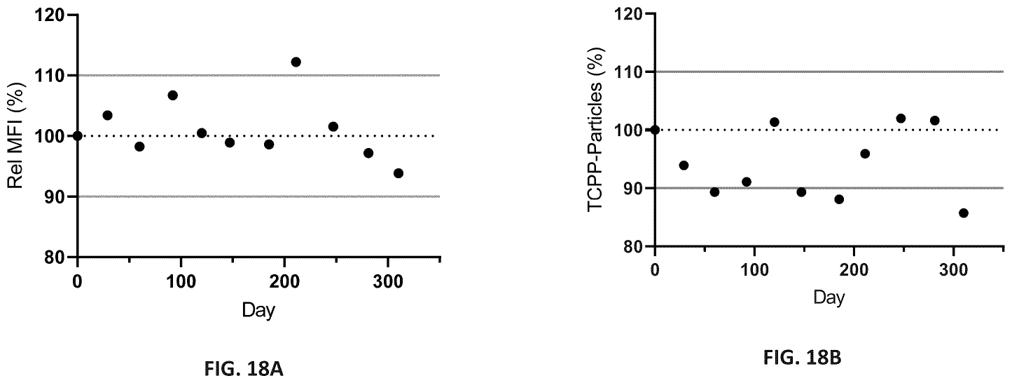

one aspect of the present invention provides for a method wherein synthetic amino functionalized beads are functionalized with TCPP in a suitable solvent with the addition of a peptide coupling reagent. The TCPP functionalized beads are stable for at least 1, 2, 3, 4, 5, 6 or more months. For example, the TCPP functionalized beads are stable for 1 year at room temperature (See FIG. 18).

### EXAMPLES

The invention is further illustrated by the following non-limiting examples.

Materials. TCPP (5,10,15,20-tetrakis-(4-carboxyphenyl)-21,23H-porphine) (Frontier Scientific, 97%) was purchased from Fisher Scientific (cat. A5015100 MG). EDC (N-(3-dimethylaminopropyl)-N′-ethyl carbodiimide hydrochloride) (≥98%) was purchased from Sigma (cat. 03450-1G). Sodium bicarbonate (ACS grade) was purchased from Sigma (cat. S6014). MES (2-(N-morpholino)-N′-ethanesulfonic acid hemisodium salt) was purchased from Sigma (cat. M0164). (Isopropanol (ACS) was purchased from Fisher Chemical (cat. A516-4). HBSS (Hank's balanced salt solution, —CaCl2, —MgCl2, —MgSO4) was purchased from Fisher Scientific (cat. 14-175-095). Purified water was from in-house system (Aqua Solutions). Amino-polystyrene particles (cross-linked) (8.0-12.9 μm diameter, 2.5% w/v) were purchased from Spherotech (cat. APX-100-10). Amino polystyrene divinylbenzene crosslinked PS/DVB beads (10 μm, 5 μm) were purchased from EPRUI Biotech (cat. 1-003-10 and 1-003-5, respectively). Glass fiber syringe filters (Finneran, 5 μm, 13 mm diameter) were purchased from Thomas Scientific (cat. 1190M60). Flow cytometry was performed on a Navios EX flow cytometer (Beckman Coulter).

### Example 1

Preparation of TCPP stock solution. In a 150 mL Pyrex beaker with a stir bar were combined purified water (25.3 mL) and isopropanol (25.3 mL) with stirring. Sodium bicarbonate (202 mg, 2.40 mmol) was added and stirred until dissolved. TCPP (50.5 mg, 0.0639 mmol) was added, and the beaker lightly covered with Parafilm and then aluminum foil to exclude light. Stirring was continued for 26 min, at which time a purple solution resulted. The solution was stored capped in a polypropylene tube at room temperature in the dark. The resultant solution was 1.26×10⁻³ M in TCPP.

### Example 2

Preparation of MES buffer solution. To a 150 mL disposable plastic bottle (Fisher Scientific, cat. 14-959-10B) was added MES hemisodium salt (2.5006 g, 0.0121 mmol) followed by 121 mL of purified water. The bottle was capped and shaken until a clear solution was obtained. The pH of the solution was measured using a standardized Denver Instruments UB-10 PH meter and found to be 6.11. This solution was stored at room temperature and used on the same day it was prepared. The solution was 0.100 M in MES.

### Example 3

Preparation of EDC solution. To a 50 mL polypropylene tube with cap (Falcon) (Fisher Scientific cat. 14-432-22) was added EDC (199.4 mg, 1.04 mmol) followed by 20.0 mL purified water and the tube briefly mixed by vortex to give a clear solution which was designated DIL 0 (5.20×10⁻² M). To a 150 mL disposable plastic bottle was added MES buffer (54.0 mL) followed by MES DIL 0 (6.00 mL, 0.312 mmol). The bottle was capped and shaken vigorously to give a clear solution (5.20×10⁻³ M). This solution was designated as DIL 1. This solution was used within 15 min of preparation. In an alternate but closely related procedure used in the TCPP-Amine bead volumetric ratio experiments, DIL 1 was prepared by addition of EDC DIL 0 (12.85 mL) to MES buffer (54.0 mL) and the mixture vigorously shaken to afford a clear solution 1.00×10⁻² M in EDC.

### Example 4

General TCPP/EDC bead staining procedure (Spherotech beads). To a 15 mL polypropylene tube (Falcon) with cap (Fisher Scientific, cat. 14-959-53A) was added 4.50 mL of MES buffer solution followed by 0.288 mL of Spherotech bead suspension which had been freshly vortexed for 60 sec. TCPP aqueous solution (1.26×10⁻³ M) (1.20 mL, 1.51×10⁻³ mol) that had been freshly vortex mixed for 60 sec. EDC (5.20×10⁻³ M) (4.50 mL, 2.34×10⁻³ mol) was then added. The total volume of bead suspension in the staining tube was 10.29 mL. The tube was placed in an inverting agitator (Thermo, cat. 88881001) at 35 rpm for over 12 hours, for example about 16 hours) in an enclosure to exclude light. The suspension was centrifuged (21° C., 1000×g, 10 min) (Eppendorf 5810 R) and the supernatant brown solution aspirated. The beads were gently resuspended in about 0.800 mL of HBSS using a P1000 autopipette and stored in an amber plastic vial.

### Example 5

Procedure for variable EDC-TCPP ratio experiments. To a 15 mL polypropylene tubes (Falcon) with cap (Fisher Scientific, cat. 14-959-53A) labeled A-H was added MES buffer in the amounts shown in Table 1, followed by 0.288 mL/tube of Spherotech bead suspension which had been freshly vortexed for 60 sec. TCPP aqueous stock solution (1.26×10⁻³ M) (1.20 mL, 1.51×10⁻³ mol) that had been freshly vortex mixed for 60 see was added to each tube. Freshly prepared EDC solution (5.20×10⁻³ M) was then added to each tube according to Table 1. The total volume of bead suspension in the staining tubes was 10.3 mL/tube. The tubes were placed in an inverting agitator (Thermo, cat. 88881001) at 35 rpm for over 12 hours for example 16-20 hours in an enclosure to exclude light. The suspension was centrifuged (21° C., 1000×g, 10 min) (Eppendorf 5810 R) and the supernatant brown solution aspirated. The beads were gently resuspended in 0.800 mL of HBSS using a P1000 autopipette and stored in amber plastic vials.

### Example 6

Procedure for variable TCPP-bead ratio experiments. To a 15 mL polypropylene tubes (Falcon) with cap (Fisher Scientific, cat. 14-959-53A) labeled A-F was added MES buffer in the amounts shown in Table 3, followed by 0.288 mL/tube of Spherotech bead suspension which had been freshly vortexed for 60 sec. TCPP aqueous stock solution (1.26×10⁻³ M) that had been freshly vortex mixed for 60 see was added to each tube per the amounts indicated in Table 3. Freshly prepared EDC solution (1.00×10⁻² M) was then added to each tube according to Table 3. The total volume of bead suspension in the staining tubes was 10.3 mL/tube. The tubes were placed in an inverting agitator (Thermo, cat. 88881001) at 35 rpm for over about 12 hours for example 16-20 hours in an enclosure to exclude light. The suspension was centrifuged (21° C., 1000×g, 10 min) (Eppendorf 5810 R) and the supernatant brown solution aspirated. The beads were gently resuspended in 0.800 mL of HBSS using a P1000 autopipette and stored in amber plastic vials.

### Example 7

General TCPP/EDC bead staining procedure (EPRUI beads). To a 15 mL polypropylene tube (Falcon) with cap (Fisher Scientific, cat. 14-959-53A) was added of MES buffer solution (5.00 mL) followed by EPRUI beads (10.0 mg). TCPP stock solution (1.26×10⁻³ M) (1.20 mL, 1.51×10⁻³ mol) that had been freshly vortex mixed for 60 sec. EDC (5.20×10⁻³ M) (4.53 mL, 2.34×10⁻³ mol) was then added. The total volume of bead suspension in the staining tube was 10.29 mL. The tube was placed in an inverting agitator (Thermo, cat. 88881001) at 35 rpm for >about 12 h in an enclosure to exclude light. The suspension was centrifuged (21° C., 1000×g, 10 min) (Eppendorf 5810 R) and the supernatant brown solution aspirated. The beads were gently resuspended in 1 mL of HBSS using a P1000 autopipette and stored in an amber plastic vial.

### Example 8

Bead staining for 5 μm EPRUI beads. To a 15 mL polypropylene tubes (Falcon) with cap (Fisher Scientific, cat. 14-959-53A) was added MES buffer (0.1 M) (5.00 mL), followed by freshly vortexed amine polystyrene beads (EPRUI Biotech, Cat. 1-003-5) (0.079 mL), TCPP stock solution (1.26×10⁻³ M aq.) (1.20 mL, 1.51×10⁻³ mmol), and EDC in MES (5.23×10⁻³ M) (4.519 mL, 2.35×10⁻² mmol). The tube was placed in an inverting agitator (Thermo, cat. 88881001) at 35 rpm for about 16 hours in an enclosure to exclude light. The bead suspension was centrifuged (21° C., 1000×g, 10 min) (Eppendorf 5810 R) and the supernatant brown solution aspirated. The beads were gently resuspended in 0.800 mL of HBSS using a P1000 autopipette and stored in an amber plastic vial.

### Example 9

TCPP NHS ester bead staining procedure. To a 15 ml conical plastic tube with cap (Falcon) was added TCPP in DMF (1.26×10⁻³ M) (1.20 mL, 1.51×10⁻³ mmol), followed by EDC (6.7 mg, 3.5×10⁻² mmol) and NHS (14.0 mg, 1.22×10⁻² mmol). The tube was briefly mixed by vortex to afford a solution that was stored under foil at room temperature for 5 hours (“h”). To the tube was then added a freshly vortexed suspension of polystyrene functionalized amine beads (Spherotech, cat APX-100-10) (0.288 mL) and the tube placed in a rotating agitator at 35 rpm for 16 h with the exclusion of light. The suspension was centrifuged (21° C., 1000×g, 10 min) (Eppendorf 5810 R) and the supernatant brown solution aspirated. The beads were gently resuspended in 0.800 mL of HBSS using a P1000 autopipette and stored in an amber plastic vial.

### Example 10

Filtration procedure. TCPP-functionalized bead suspension was vortex mixed (high) for 60 sec. The bead suspension was diluted 4× by transferring an aliquot (0.800 mL) to a 5.00 mL plastic tube, followed by HBSS (3.20 mL). The tube was capped and mixed by vortex (high) for 15 sec. The plunger was removed from a 5 mL disposable syringe, was fitted with a filter, and held by a clamp (filter down). To the syringe was added HBSS (4.00 mL). The diluted parent bead suspension (0.500 mL) was added to the syringe and the resultant suspension mixed in the syringe using a P1000 autopipette. The syringe plunger was re-attached, and the bead suspension filtered by gently pushing the plunger to achieve a filtration rate of 1-10 drops/see, collecting the filtrate in a 15 mL conical tube. Fresh HBSS (5.0 mL) was slowly drawn back through the filter into the syringe and ten pushed out through the filter as before. The combined filtrates were saved for further analysis. Beads were removed from the filter by first slowly drawing fresh HBSS (5.0 mL) through the filter into the syringe, removing the filter, and dispensing the HBSS into a 50 ml conical tube. This was repeated a total of five times (total HBSS 25 mL). The conical tube was protected from light when not in use. All tubes (diluted unfiltered bead suspensions, filtered bead suspensions, filtrates) were centrifuged (21° C., 1000×g, 10 min) and the supernatant liquids discarded. The pellets were resuspended as follows: bead suspensions in HBSS (0.500 mL/tube), diluted unfiltered bead suspensions in HBSS (0.800 mL/tube), particulates from filtrate in HBSS (0.500 mL/tube). Resuspension was done using a P1000 autopipette. All samples were dispensed to 2 mL amber tubes and labeled.

### Example 11

Functionalization of 10.6 μm amine polystyrene beads by the TCPP-NHS monoester method. To a 15 ml conical plastic tube with cap (Falcon) was added TCPP (61.5 mg, 7.8×10⁻² mmol) followed by NHS (9.3 mg, 8.1×10⁻² mmol). DMF (anhydrous) (10.0 mL) was then added and the tube placed in a rotating agitator (Thermo) at 35 rpm at room temperature for 4 h, protected from light. A separate 15 mL plastic tube was charged with DMF (9.80 mL) followed by freshly vortexed amine functionalized polystyrene beads (Spherotech, Cat. APX-100-10) (0.288 mL) and then the TCPP solution (0.190 mL, 1.48×10−3 mmol) and the tube placed in the rotating agitator as above for 15 h. The suspension was centrifuged (21° C., 1000×g, 10 min) (Eppendorf 5810 R) and the supernatant brown solution aspirated. The beads were gently resuspended in 0.800 mL of HBSS using a P1000 autopipette and stored in an amber plastic vial.

### Example 12

Functionalization of 10.6 μm amine polystyrene beads by the TCPP-NHS monoester method with added EDC. To a 15 mL conical plastic tube with cap (Falcon) was a stock solution of TCPP (1.26×10⁻³ M in DMF) (1.20 mL, 1.51×10−3 mmol), followed by a stock solution of EDC (1.26×10⁻³ M in DMF), (1.20 mL, 1.51×10−3 mmol), and a stock solution of NHS (1.26×10⁻³ M in DMF), (1.20 mL, 1.51×10⁻³ mmol) and the mixture vortex mixed to afford a purple solution. This was stored at room temperature for 2 h. At this time, freshly vortexed amine functionalized polystyrene beads (Spherotech, Cat. APX-100-10) (0.288 mL) were added and the tube placed in the rotating agitator as above for 19 h. The suspension was centrifuged (21° C., 1000×g, 10 min) (Eppendorf 5810 R) and the supernatant brown solution aspirated. The beads were gently resuspended in 0.800 mL of HBSS using a P1000 autopipette and stored in an amber plastic vial.

### Example 13

Functionalization of 8.66 μm beads are described according to the following example. To a 15 mL conical tube with cap (CELLTREAT) was added MES buffer (4.30 mL), followed by 0.288 mL of bead suspension (Spherotech, Cat. APX-100-10, Lot AF01, 8.66 μm diameter) which had been freshly vortexed for 60 sec. TCPP aqueous stock solution (1.26×10⁻³ M) (1.20 mL, 1.51×10⁻³ mmol), followed by EDC (5.20×10⁻³ M) (4.50 mL, 2.34×10⁻³ mol) and the tubes placed in the rotating agitator as above for 19 hour. The suspension was centrifuged (21° C., 1000×g, 10 min) (Eppendorf 5810 R) and the supernatant brown solution aspirated. The beads were gently resuspended in 0.800 mL of HBSS using a P1000 autopipette and stored in an amber plastic vial. The MFI of these beads was 575.

### Example 14

Reaction between TCPP and EDC in the absence of beads. To each of three 15 mL conical tubes with caps (FALCON) was added MES buffer (4.30 mL), followed by 0.288 mL additional MES buffer, a volume equivalent to what would have been added of the bead suspension. TCPP aqueous stock solution (1.26×10⁻³ M) (1.20 mL, 1.51×10⁻³ mmol), followed by EDC (5.20×10⁻³ M) (4.50 mL, 2.34×10⁻³ mol) and the tubes placed in the rotating agitator as above for 18 h. The suspension was centrifuged (21° C., 1000×g, 10 min) (Eppendorf 5810 R) and the supernatant brown solution aspirated. The beads were gently resuspended in 0.800 mL of HBSS using a P1000 autopipette and stored in an amber plastic vial.

### Example 15

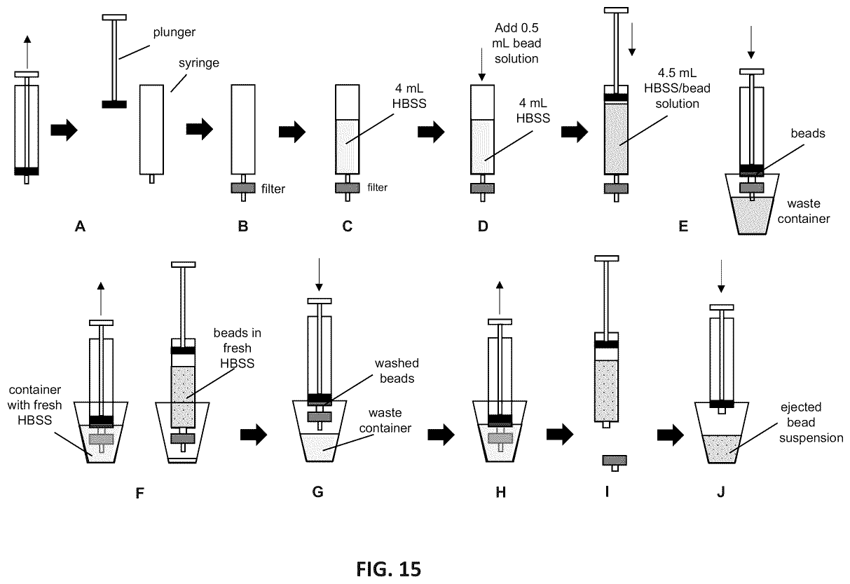

Reaction between TCPP and beads without EDC. To each of three 15 ml conical tubes with caps (FALCON) was added MES buffer (4.30 mL), followed by 0.288 mL of 10.6 μm amine beads (Spherotech, Cat. APX-100-10). TCPP aqueous stock solution (1.26×10⁻³ M) (1.20 mL, 1.51×10⁻³ mmol), followed by EDC (5.20×10⁻³ M) (4.50 mL, 2.34×10⁻³ mol) and the tubes placed in the rotating agitator as above for 20 h. The suspension was centrifuged (21° C., 1000×g, 10 min) (Eppendorf 5810 R) and the supernatant brown solution aspirated. The beads were gently resuspended in 0.800 mL of HBSS using a P1000 autopipette and stored in an amber plastic vial. (See FIG. 15 for flow chart of one embodiment)

### Example 16

Variable bead staining time. If the amine functionalized beads were labeled for a longer period of time with TCPP, the amount of particulates increased as compared to labeling with TCPP for a shorter period of time less particulate formation resulted.

### Example 17

Functionalization of 10.6 μm amine beads (Spherotech) with TCPP and EDC in MES at low temperature. All solutions and suspensions were cooled to 2-6° C. prior to use and kept at this temperature throughout the reaction. To a 15 mL polypropylene tube (Falcon) with cap was added 4.30 mL of MES buffer solution followed by 0.288 mL of Spherotech bead suspension which was freshly vortexed for 30 sec. TCPP aqueous solution (1.26×10⁻³ M) (1.20 mL, 1.51×10⁻³ mol) that was freshly vortex mixed for 30 sec. EDC (5.20×10⁻³ M) (4.50 mL, 2.34×10⁻³ mol) was then added. The total volume of bead suspension in the staining tube is 10.3 mL. The tube is placed in an inverting agitator (Thermo, cat. 88881001) at 35 rpm for over 12 hours, for example about 22 h at 6° C. in an enclosure to exclude light. The suspension was centrifuged (21° C., 1000×g, 10 min) (Eppendorf 5810 R) and the supernatant brown solution aspirated. The beads were gently resuspended in 0.800 mL of HBSS using a P1000 autopipette and are stored in an amber plastic vial. MFI was 870. Frequency of parent for positive bead population was 57.4% and frequency of parent for TCPP-related particulates was 38.4%.

### Example 18

Functionalization of 10.6 μm amine beads (Spherotech) with TCPP, NHS, and EDC with pre-filtration. To a 15 ml conical plastic tube with cap (Falcon) was added EDC (21.5 mg, 1.21×10⁻¹ mmol) and NHS (40.7 mg, 3.54×10⁻¹ mmol), then TCPP in DMF (1.26×10⁻³ M) (1.20 mL, 1.51×10⁻³ mmol). The tube was mixed by vortex (high) for 30 see to afford a solution that was stored under foil at room temperature for 5 h. To the solution is added purified water (1.20 mL) and the resultant cloudy mixture is filtered through a filter (5 μm porosity) and the filtrate placed in a fresh 15 ml conical tube. To the tube was then added a freshly vortexed suspension of polystyrene functionalized amine beads (Spherotech, cat APX-100-10) (0.288 mL) and the tube was placed in a rotating agitator at 35 rpm for 16 h with the exclusion of light. The suspension is centrifuged (21° C., 1000×g, 10 min) (Eppendorf 5810 R) and the supernatant brown solution aspirated. The beads are gently resuspended in 0.800 mL of HBSS using a P1000 autopipette and are stored in an amber plastic vial. MFI was 906. Frequency of parent for positive bead population was 85.6% and frequency of parent for TCPP-related particulates was 6.11%

### Example 19

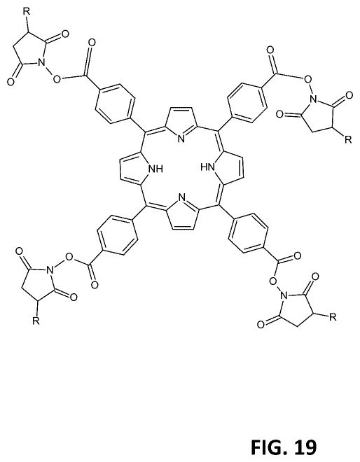

TCPP Tetra-NHS ester is illustrated in FIG. 19 for use in a further example.

### Example 20

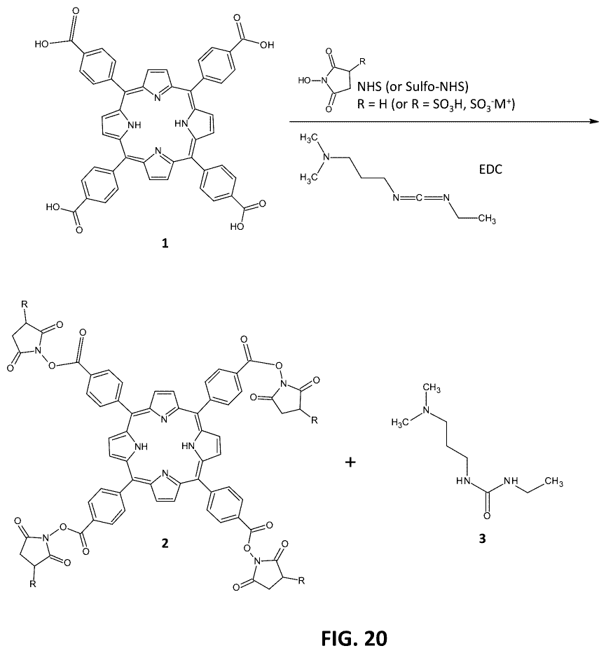

TCPP Sulfo-NHS ester bead staining procedure. To a 15 ml conical plastic tube with cap (Falcon) is added TCPP in DMF (1.26×10⁻³ M) (1.20 mL, 1.51×10⁻³ mmol), followed by EDC (6.7 mg, 3.5×10⁻² mmol) and Sulfo-NHS (hydroxy-2,5-dioxopyrrolidine-3-sulfonicacid sodium salt) (2.65 mg, 1.22×10⁻² mmol) as is illustrated according to FIG. 20 according to one embodiment of the present invention. The tube is briefly mixed by vortex to afford a solution that is stored under foil at room temperature for 2-6 h. To the tube, a freshly vortexed suspension of polystyrene functionalized amine beads (Spherotech, cat APX-100-10) (0.288 mL) is added and the tube is placed in a rotating agitator at 35 rpm for 16 h with the exclusion of light. The suspension is centrifuged (21° C., 1000×g, 10 min) (Eppendorf 5810 R) and the supernatant brown solution aspirated. The beads are gently resuspended in 0.800 mL of HBSS using a P1000 autopipette and stored in an amber plastic vial.

### Example 21

Functionalization of amine-functionalized beads with TCPP and EDC in neutral buffered solution is described according to one embodiment of the present invention as follows: To a 15 mL polypropylene tube (Falcon) with cap was added 4.30 mL of Dubelco's phosphate buffered saline (DPBS) buffer solution followed by 0.288 mL of Spherotech bead suspension which is freshly vortexed for 60 sec. TCPP aqueous solution (1.26×10⁻³ M) (1.20 mL, 1.51×10⁻³ mol) that was freshly vortex mixed for 60 sec. EDC (5.20×10⁻³ M in DPBS) (4.50 mL, 2.34×10⁻³ mol) was then added. The total volume of bead suspension in the staining tube was 10.3 mL. The tube was placed in an inverting agitator (Thermo, cat. 88881001) at 35 rpm for 17 h (over 12 h) at ambient temperature in an enclosure to exclude light. The suspension was centrifuged (21° C., 1000×g, 10 min) (Eppendorf 5810 R) and the supernatant brown solution aspirated. The beads were gently resuspended in 0.800 mL of HBSS using a P1000 autopipette and are stored in an amber plastic vial. MFI was 1102. Frequency of parent for positive bead population was 84.8% and frequency of parent for TCPP-related particulates was 9.64%.

The preceding examples can be repeated with similar success by substituting the generically or specifically described reactants and/or operating conditions of this invention for those used in the preceding examples.

Note that in the specification and claims, “a”, “an”, and “the” includes the singular and the plural and should be read as “one or more” of the element(s) preceded by the article “a”, “an” and “the” unless otherwise indicated by the context in which used; “about” or “approximately” means within twenty percent (20%) of the numerical amount cited. All computer software disclosed herein may be embodied on any computer-readable medium (including combinations of mediums), including without limitation CD-ROMs, DVD-ROMs, hard drives (local or network storage device), USB keys, other removable drives, ROM, and firmware.

Although the invention has been described in detail with particular reference to these embodiments, other embodiments can achieve the same results. For example, the MFI of the TCPP functionalized beads may be tuned based upon the reaction conditions to achieve the preferred range depending on the use to which the calibration bead is applied for example compensation. Also, the percentage of TCPP particulates can be similarly tuned in the preparation of the beads. Further another aspect of an embodiment of the present invention is adjusting the MFI of the amine functional beads bonded with TCPP for example to keep the TCPP fluorescence of the beads within the range of a detector on a given instrument or to make the bead fluorescence similar to that of a stained cell population that is of interest from the standpoint of compensation. Variations and modifications of the present invention will be obvious to those skilled in the art and it is intended to cover in the appended claims all such modifications and equivalents. The entire disclosures of all references, applications, patents, and publications cited above are hereby incorporated by reference.

### BIBLIOGRAPHY

(1) Böhmer, R. M.; Morstyn, G. Uptake of Hematoporphyrin Derivative by Normal and Malignant Cells: Effect of Serum, PH, Temperature, and Cell Size. Cancer research 1985, 45 (11 Part 1), 5328-5334.
(2) Mohamed Al-Far; Neville Pimstone. A Comparative Study of 28 Porphyrins and Their Abilities to Localize in Mouse Mammary Carcinoma: Uroporphyrin I Superior to Hematoporphyrin Derivative. Prog Clin Biol Res 1984, 170, 661-672.
(3) Boyle, R. W.; Dolphin, D. Structure and Biodistribution Relationships of Photodynamic Sensitizers. Photochemistry and Photobiology 1996, 64 (3), 469-485. https://doi.org/10.1111/j.1751-1097.1996.tb03093.x.
(4) Brown, S. B.; Brown, E. A.; Walker, I. The Present and Future Role of Photodynamic Therapy in Cancer Treatment. Lancet Oncol. 2004, 5 (8), 497-508. https://doi.org/10.1016/S1470-2045(04)01529-3.
(5) Dougherty, T. J.; Gomer, C. J.; Henderson, B. W.; Jori, G.; Kessel, D.; Korbelik, M.; Moan, J.; Peng, Q. Photodynamic Therapy. J Natl Cancer Inst 1998, 90 (12), 889-905. https://doi.org/10.1093/jnci/90.12.889.
(6) Hahn, S. M.; Putt, M. E.; Metz, J.; Shin, D. B.; Rickter, E.; Menon, C.; Smith, D.; Glatstein, E.; Fraker, D. L.; Busch, T. M. Photofrin Uptake in the Tumor and Normal Tissues of Patients Receiving Intraperitoneal Photodynamic Therapy. Clinical Cancer Research 2006, 12 (18), 5464-5470. https://doi.org/10.1158/1078-0432.CCR-06-0953.
(7) de Visscher, S. A. H. J.; Dijkstra, P. U.; Tan, I. B.; Roodenburg, J. L. N.; Witjes, M. J. H. MTHPC Mediated Photodynamic Therapy (PDT) of Squamous Cell Carcinoma in the Head and Neck: A Systematic Review. Oral Oncology 2013, 49 (3), 192-210. https://doi.org/10.1016/j.oraloncology.2012.09.011.
(8) Usuda, J.; Kato, H.; Okunaka, T.; Furukawa, K.; Tsutsui, H.; Yamada, K.; Suga, Y.; Honda, H.; Nagatsuka, Y.; Ohira, T.; others. Photodynamic Therapy (PDT) for Lung Cancers. Journal of Thoracic Oncology 2006, 1 (5), 489-493.
(9) Hamblin, R.; Luke, E. On the Mechanism of the Tumour-Localising Effect in Photodynamic Therapy. 6.
(10) Hamblin, M. R.; Newman, E. L. New Trends in Photobiology: On the Mechanism of the Tumour-Localising Effect in Photodynamic Therapy. Journal of Photochemistry and Photobiology B: Biology 1994, 23 (1), 3-8.
(11) Tsolekile, N.; Nelana, S.; Oluwafemi, O. S. Porphyrin as Diagnostic and Therapeutic Agent. Molecules 2019, 24 (14). https://doi.org/10.3390/molecules24142669.
(12) Patriquin, L.; Merrick, D. T.; Hill, D.; Holcomb, R. G.; Lemieux, M. E.; Bennett, G.; Karia, B.; Rebel, V. I.; Bauer, T. Early Detection of Lung Cancer with Meso Tetra (4-Carboxyphenyl) Porphyrin-Labeled Sputum. J Thorac Oncol 2015, 10 (9), 1311-1318. https://doi.org/10.1097/JTO.0000000000000627.
(13) Maciorowski, Z.; Chattopadhyay, P. K.; Jain, P. Basic Multicolor Flow Cytometry. Curr Protoc Immunol 2017, 117, 5.4.1-5.4.38. https://doi.org/10.1002/cpim.26.
(14) Roederer, M. Compensation in Flow Cytometry. Current Protocols in Cytometry 2002, 22 (1), 1.14.1-1.14.20. https://doi.org/10.1002/0471142956.cy0114s22.
(15) Szalóki, G.; Goda, K. Compensation in Multicolor Flow Cytometry. Cytometry Part A 2015, 87 (11), 982-985. https://doi.org/10.1002/cyto.a.22736.
(16) Grayson, M.; Lai, S.-C.; Bederka, L. H.; Araujo, P.; Sanchez, J.; Reveles, X. T.; Rebel, V. I.; Rebeles, J. Quality-Controlled Sputum Analysis by Flow Cytometry. JoVE (Journal of Visualized Experiments) 2021, No. 174, e62785. https://doi.org/10.3791/62785.
(17) Pasternack, R. F.; Huber, P. R.; Boyd, P.; Engasser, G.; Francesconi, L.; Gibbs, E.; Fasella, P.; Venturo, G. C.; Hinds, L. de C. On the Aggregation of Meso-Substituted Water-Soluble Porphyrins. J. Am. Chem. Soc. 1972, 94 (13), 4511-4517.
(18) Siggel, U.; Bindig, U.; Endisch, C.; Komatsu, T.; Tsuchida, E.; Voigt, J.; Fuhrhop, J.-H. Photophysical and Photochemical Properties of Porphyrin Aggregates. Berichte der Bunsengesellschaft für physikalische Chemie 1996, 100 (12), 2070-2075. https://doi.org/10.1002/bbpc.19961001225.
(19) Kabe, Y.; Ohmori, M.; Shinouchi, K.; Tsuboi, Y.; Hirao, S.; Azuma, M.; Watanabe, H.; Okura, I.; Handa, H. Porphyrin Accumulation in Mitochondria Is Mediated by 2-Oxoglutarate Carrier. J Biol Chem 2006, 281 (42), 31729-31735. https://doi.org/10.1074/jbc.M604729200.
(20) Shimizu, N.; Sugimoto, K.; Tang, J.; Nishi, T.; Sato, I.; Hiramoto, M.; Aizawa, S.; Hatakeyama, M.; Ohba, R.; Hatori, H.; Yoshikawa, T.; Suzuki, F.; Oomori, A.; Tanaka, H.; Kawaguchi, H.; Watanabe, H.; Handa, H. High-Performance Affinity Beads for Identifying Drug Receptors. Nat Biotechnol 2000, 18 (8), 877-881. https://doi.org/10.1038/78496.
(21) Ohtsu, Y.; Ohba, R.; Imamura, Y.; Kobayashi, M.; Hatori, H.; Zenkoh, T.; Hatakeyama, M.; Manabe, T.; Hino, M.; Yamaguchi, Y.; Kataoka, K.; Kawaguchi, H.; Watanabe, H.; Handa, H. Selective Ligand Purification Using High-Performance Affinity Beads. Anal Biochem 2005, 338 (2), 245-252. https://doi.org/10.1016/j.ab.2004.10.006.
(22) Anderson, G. W.; Zimmerman, J. E.; Callahan, F. M. The Use of Esters of N-Hydroxysuccinimide in Peptide Synthesis. J. Am. Chem. Soc. 1964, 86 (9), 1839-1842. https://doi.org/10.1021/ja01063a037.
(23) El-Faham, A.; Albericio, F. Peptide Coupling Reagents, More than a Letter Soup. Chem. Rev. 2011, 111 (11), 6557-6602. https://doi.org/10.1021/cr100048w.

What is claimed is:

1. A compensation bead for use in a flow cytometer comprising:
an amine functionalized polymer bead having a diameter of between about 3 um to about 18 um wherein an amine group is covalently bonded to the exterior surface of the polymer bead; and
a carboxyaryl porphyrin bonded to the exterior surface of the amine functionalized polymer bead via-a non-covalent bond and via a covalent bond with the amine group in such a manner that when the amine functionalized polymer bead bonded with the carboxyaryl porphyrin is suspended in a media, a fluorescence spectra exhibited by the amine functionalized polymer bead bonded with the carboxyaryl porphyrin is comparable with the fluorescence spectra of the carboxyaryl porphyrin

2. The compensation method of claim 1 wherein the carboxyaryl porphyrin is bonded covalently to the amine functionalized bead via an amide bond whereby the carboxy portion is present in the carboxyaryl porphyrin and the amine portion is part of the amine functionalized bead in the absence of a linker.

3. The compensation bead of claim 1 wherein the amine functionalized bead is formed of a polymer selected from polystyrene, divinylbenzene crosslinked polystyrene, latex, polyvinylamine (PVA), chitosan, poly(methyl methacrylate) (PMMA), epoxy, silica, carbon fiber, and iron oxide.

4. The compensation bead of claim 1 wherein the diameter of the amine functionalized bead is between about 5 μm to about 15 μm.

5. The compensation bead of claim 1 wherein the media is selected from Dubelco's phosphate buffered saline, (DPBS), Hank's balanced salt solution (HBBS) or a combination thereof in a pH range of between about 6.5 to about 7.5.

6. The compensation bead of claim 1 wherein the fluorescence spectra has an excitation of about 400 nm and an emission of between about 600 nm to about 800 nm.

7. The compensation bead of claim 1 wherein there is no linker covalently connecting a carboxyaryl porphyrin carboxamide and the surface of the polymer bead.

8. The compensation bead of claim 1 wherein there is no linker covalently connecting the carboxyaryl porphyrin carboxamide and the surface of the polymer bead.

9. A method for making a compensation bead of claim 1 comprising:
reacting an amine group on a polymer bead surface with a carboxyaryl porphyrin having an activated ester to produce a carboxaryl porphyrin carboxamide functionalized bead;
separating an unreacted carboxyaryl porphyrin from the carboxaryl porphyrin carboxamide functionalized bead; and
collecting the carboxaryl porphyrin carboxamide functionalized bead for use as the compensation bead.

10. The method of claim 9 wherein the unreacted carboxyaryl porphyrin comprises carboxyaryl porphyrin in a solution of the reacting step, carboxyaryl porphyrin derivatives formed during the reacting step or a combination thereof.

11. The method of claim 9 wherein a bond of the amine group of the polymer bead surface with carboxyaryl porphyrin is a covalent bond, a non-covalent bond or a combination thereof.

12. The method of claim 9 further comprising incubating the carboxyaryl porphyrin with N-(3-dimethylaminopropyl)-N′-ethyl carbodiimide hydrochloride (EDC), N-hydroxysuccinimide (NHS), sulfo-NHS or any combination thereof in dimethylformamide (DMF) to produce the carboxyaryl porphyrin with the activated ester prior to the reacting step.

13. The method as in claim 12 wherein the carboxyaryl porphyrin with the activated ester is in a DMF solution and combined with water and filtered to produce a filtrate of the carboxyaryl porphyrin with activated ester prior to the reaction step.

14. The method of claim 9 wherein the EDC and the carboxyaryl porphyrin are present at a mol/mol ratio of between about 0.35 to about 25.91.

15. The method of claim 9 wherein the reacting step proceeds in a solution comprising at least one of 2-(N-morpholino)-N′-ethanesulfonic acid hemisodium salt (MES) DBPS, DMF, water, aqueous DMF or any combination thereof at a pH of between about 6 to about 7.

16. The method of claim 9 wherein the compensation beads have a median fluorescence intensity of between about 140 to about 1500.

17. The method of claim 9 wherein the composition of the bead is selected from polystyrene, divinylbenzene crosslinked polystyrene, latex, polyvinylamine (PVA), chitosan, poly(methyl methacrylate) (PMMA), epoxy, silica, carbon fiber, and iron oxide.

18. The method of claim 9 wherein a diameter of the compensation bead is between about 5 μm to about 20 μm.

20. The method of claim 9 wherein the carboxaryl porphyrin carboxamide functionalized beads have a fluorescence spectra with an excitation of about 400 nm and an emission of between about 600 nm to about 800 nm.

21. The method of claim 9 wherein there is no linker on the polymer bead surface.

22. The method of claim 9 wherein the separating step comprises
filtering the unreacted carboxyaryl porphyrin and the carboxaryl porphyrin carboxamide functionalized beads through a filter of about 5 μm porosity; and
removing the carboxaryl porphyrin carboxamide functionalized beads from the filter.

23. A method for making a compensation bead of claim 1 comprising:
reacting an amine present on a polymer bead surface that forms an amine functionalized bead with a carboxyaryl porphyrin and EDC in a solution having one or more selected from MES buffer (pH about 6), DPBS buffer (pH about 7), DMF, and water to produce a carboxaryl porphyrin carboxamide functionalized bead;
separating an unreacted carboxyaryl porphyrin from the amine functionalized bead bonded with carboxyaryl porphyrin; and
collecting the carboxaryl porphyrin carboxamide functionalized bead for use as the compensation bead.
